SEQUENCE LISTING (5 sequences)

SEQ ID NO: 1 - protein, 5 aa, synthetic construct
IKVAV

SEQ ID NO: 2 - protein, 5 aa, synthetic construct
YIGSR

SEQ ID NO: 3 - protein, 7 aa, synthetic construct
GRGDSPC

SEQ ID NO: 4 - protein, 6 aa, synthetic construct
  MOD_RES 3: note = hydroxyproline
GFPGER

SEQ ID NO: 5 - protein, 16 aa, synthetic construct
GCRDVPMSMRGGDRCG

(19) United States
(12) Patent Application Publication
(10) Pub. No.: US 2025/0136927 A1
(43) Pub. Date: May 1, 2025

(54) COMPOSITION FOR 3D TISSUE CULTURE

(71) Applicants: The University Court of the University of Glasgow, Glasgow Strathclyde (GB); Imperial College Innovations Limited, London (GB); Cellink Bioprinting AB, Gothenburg (SE)

(72) Inventors: Oana DOBRE, Glasgow Strathclyde (GB); Sara TRUJILLO-MUNOZ, Saarbrucken (DE); Matthew John DALBY, Glasgow Strathclyde (GB); Manuel SALMERON-SANCHEZ, Glasgow Strathclyde (GB); Liliang OUYANG, Beijing (CN); Molly STEVENS, London (GB); Adel Itedal Namro REDWAN, Gothenburg (SE); Volodymyr KUZMENKO, Gothenburg (SE); Erik GATENHOLM, Gothenburg (SE); Hector MARTINEZ, Gothenburg (SE)

(73) Assignees: The University Court of the University of Glasgow, Glasgow Strathclyde, GB (GB); Imperial College Innovations Limited, London, GB (GB); Cellink Bioprinting AB, Gothenburg (SE)

(21) Appl. No.: 18/691,266

(22) PCT Filed: Sep. 13, 2022
(86) PCT No.: PCT/EP2022/075418

§ 371 Date: Mar. 12, 2024

(30) Foreign Application Priority Data
Sep. 14, 2021 (GB) 2113077.8

(51) Int. Cl. C12N 5/00; C12N 5/071; C12N 5/077

(52) U.S. Cl. CPC C12N 5/0062; C12N 5/0656; C12N 5/0691; C12N 2513/00; C12N 2533/30; C12N 2533/52; C12N 2533/90; C12N 2537/10

(57) ABSTRACT

The invention relates to a composition for preparing a 3D scaffold for culturing cells and tissue, such as human cells and tissue. In particular embodiments, the present invention relates to a composition comprising a biocompatible polymer suitable for the preparation of a hydrogel, and a modified extracellular matrix (ECM) protein that is unreactive towards the biocompatible polymer, such that, after preparation of a hydrogel, the modified ECM protein is not covalently bound to the hydrogel. Compositions of the invention are suitable for use in 3D bioprinting, tissue engineering, drug screening, disease modelling and methods of treatment such as tissue regeneration.

### PRIORITY

This application is a 371 National Stage filing and claims the benefit under 35 U.S.C. § 120 of International Application No. PCT/EP2022/075418, filed 13 Sep. 2022, which claims priority to Great Britain Application No. GB 2113077.8 filed on 14 Sep. 2021, the contents and elements of which are herein incorporated by reference for all purposes.

### SEQUENCE LISTING INCORPORATION BY REFERENCE

The application herein incorporates by reference in its entirety the sequence listing material submitted concurrently with the specification as an XML file, with a filename of “to EPO-Sequence Listing [8320046]”, a creation date of Nov. 24, 2022, and a size of 8192 bytes. The ST26 Sequence Listing is part of the specification and is incorporated in its entirety by reference herein.

### FIELD OF THE INVENTION

The present invention relates to a composition for preparing a 3D scaffold for culturing cells and tissue, such as human cells and tissue. In particular, the present invention relates to a composition comprising a biocompatible polymer and a modified extracellular matrix (ECM) protein. Compositions of the invention are suitable for use in 3D bioprinting, tissue engineering, drug screening, disease modelling and methods of treatment such as tissue regeneration.

### BACKGROUND

3D scaffolds comprising synthetic or semi-synthetic polymers such as polyethylene glycol (PEG) or gelatin methacryloyl (GelMA) are commonly used to grow cells and tissues for tissue engineering applications. The final scaffolds typically comprise a highly hydrated matrix of cross-linked polymer chains that mimics the 3D network observed in the native extracellular matrix (ECM). However, the native extracellular matrix is complex, and contains many different components including structural proteins such as collagens, elastins, laminins (LM) and fibronectin (FN); glycosaminoglycans such as heparin sulfate, chondroitin sulfate, keratan sulfate and hyaluronic acid; and soluble molecules such as growth factors and cytokines. Each of these components may influence cell growth and behaviour in the native extracellular matrix. Accordingly, 3D scaffolds comprising only synthetic or semi-synthetic polymers provide a poor environment for cell or tissue growth.

Alternatively, 3D scaffolds may be prepared from extracellular matrix-derived materials such as Matrigel. However, naturally-derived materials typically have significant batch-to-batch variability, poorly defined composition and lack of controlled physical properties (such as mechanical strength or stiffness). In addition, the clinical use of such materials is complicated as they may include ill-defined additional components due to their isolation from a natural biological source. Therefore, there is a need to develop rationally designed synthetic or semi-synthetic matrices with controlled properties and composition that can more-closely mimic the native extracellular matrix environment.

High molecular weight glycoproteins, such as laminin and fibronectin, play an important role in the extracellular matrix. They have a structural role, contributing to the architecture of the extracellular matrix, they influence cell adhesion, and they bind numerous biological molecules thus influencing cell behaviour. They are also involved in matrix remodelling and, therefore, tissue homeostasis. Importantly, these proteins bind to and sequester several important growth factors (GFs), and can facilitate their interaction with other extracellular matrix components and cell-secreted molecules to support cell differentiation, migration or proliferation. Accordingly, to reproduce the extracellular matrix properties in a synthetic system, it may be beneficial to incorporate high molecular weight glycoproteins such as laminin and fibronectin.

Native (unmodified) fibronectin has been incorporated into synthetic scaffolds derived from polyethylene glycol (Trujillo et al., 2019) and hyaluronic acid methacrylate (Seidlits et al., 2011; Trujillo et al., 2020). However, the native protein was rapidly released from the scaffold. PEG hydrogels into which native fibronectin was encapsulated (i.e., simply trapped) released after 24 hours (Trujillo et al., 2019). Moreover, cells tended to migrate to the outer perimeter of the hydrogels, most likely following the path of fibronectin diffusion into the surrounding culture medium (Seidlits et al., 2011).

In order to avoid this problem, isolated extracellular matrix components, such as isolated fibronectin or laminin, have been derivatised and covalently bound to the scaffold material. Full length fibronectin has been derivatised with PEG chains having a maleimide terminal group, and this modified fibronectin has been covalently bound to a polymer network comprising PEG dithiol (Trujillo et al. 2019). The full-length fibronectin retained its key biological activities, and the resulting scaffold was shown to sequester vascular endothelial growth factor (VEGF) from the environment to promote vascularisation. When the growth factor bone morphogenetic protein (BMP2) was included, the scaffold was shown to promote bone growth in a non-healing mouse bone defect model at low BMP2 concentrations.

A similar system, in which fibronectin or fibronectin fragments were derivatised with acrylate terminated PEG and covalently bound to a polymer network comprising PEG diacrylate, has also been shown to promote osteoneogenesis, chondrogenesis and neurite outgrown (US 2006/0233855 A1). Fibronectin derivatised with acrylate terminated PEG has also been covalently incorporated into a hyaluronic acid methacrylate scaffold (Seidlits et al. 2011). Human umbilical vein endothelial cells (HUVECs) were shown to adhere, proliferate, migrate and assume an angiogenic phenotype throughout the 3D scaffold environment.

The laminin isoform LM111 has also been derivatised with acrylate terminated PEG and covalently incorporated into a PEG diacrylate scaffold (Francisco et al., 2014). The LM111 containing scaffolds were found to influence nucleus pulposus cell metabolism and expression of proposed phenotypic markers such as of N-cadherin and cytokeratin 8. Different laminin isoforms have also been derivatised and covalently incorporated into PEG hydrogels, and these have been shown to trigger stem cell differentiation towards osteogenic lineages and to stimulate growth of neural cells (Dobre et al., 2021).

However, there is an outstanding need for 3D scaffolds that can provide increased cell viability and support tissue development, and which also possess chemical and mechanical properties that make them suitable for modern scaffold fabrication techniques such as 3D bioprinting.

### SUMMARY OF THE INVENTION

The present inventors have found that a 3D scaffold providing excellent cell viability can be obtained by entrapping modified extracellular matrix proteins, such as fibronectin or laminin, within the scaffold material without covalent cross-linking. The scaffold provides excellent retention of the extracellular matrix proteins, which retain their biological function and can act as a reservoir for growth factors.

The scaffold can be prepared using 3D printing techniques, such as 3D bioprinting, as the viscosity of the compositions used to prepare the scaffold can be easily tuned by altering the quantity of modified extracellular matrix protein incorporated into the composition.

The scaffold can also be produced using purified human recombinant proteins, removing the need to screen for pathogens as occurs when using Matrigel matrices. Thus, the scaffold can be used for therapeutic applications such as tissue grafts and wound repair.

As a synthetic system, the scaffold does not suffer from batch-to-batch variability and can be used for cell and tissue growth with highly reproducible results.

Accordingly, in a first aspect of the invention there is provided a composition for use in 3D tissue culture comprising:
(a) a biocompatible polymer suitable for the preparation of a hydrogel selected from polypeptides, polysaccharides and synthetic polymers; and
(b) a modified extracellular matrix (ECM) protein, or a fragment thereof, comprising a group:

-L-B
wherein -L- is a synthetic linker and —B is a blocking group.

The blocking group does not react with the biocompatible polymer during polymerisation, and so the modified ECM protein is not covalently bound to the hydrogel. Instead, the modified ECM protein is entrapped within the hydrogel.

In a preferred embodiment the biocompatible polymer is a polypeptide, such as gelatin, such as gelatin methacryloyl (GelMA).

In a preferred embodiment the synthetic linker -L- comprises a polyethylene glycol (PEG), such as 4-arm PEG.

In a preferred embodiment the blocking group —B is a maleimide:

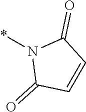 [SMILES: *N1C(=O)C=CC1=O]

where * represents the attachment point with the synthetic linker -L-.

In a second aspect of the invention there is provided a 3D scaffold obtained or obtainable by polymerisation of the composition of the first aspect.

In a third aspect of the invention there is provided a scaffold for use in 3D tissue culture comprising:
(a) a hydrogel obtained or obtainable by polymerisation of a biocompatible polymer selected from polypeptides, polysaccharides and synthetic polymers; and
(b) a modified extracellular matrix (ECM) protein, or a fragment thereof, comprising a group:

-L-B
wherein -L- is a synthetic linker and —B is a blocking group.

As in the first aspect of the invention, the blocking group does not react with the biocompatible polymer during polymerisation, and so the modified ECM protein is not covalently bound to the hydrogel. Instead, the modified ECM protein is entrapped within the hydrogel.

In a preferred embodiment the hydrogel is a polypeptide-based hydrogel, such as gelatin-based hydrogel, such as a GelMA-based hydrogel.

In a preferred embodiment the synthetic linker -L- comprises PEG, such as 4-arm PEG.

In a preferred embodiment the blocking group —B is a maleimide:

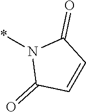 [SMILES: *N1C(=O)C=CC1=O]

where * represents the attachment point with the synthetic linker -L-.

In a fourth aspect of the invention there is provided a method for preparing a scaffold for use in 3D tissue culture, the method comprising:
(a) providing a composition of the first aspect;
(b) extruding the composition through a print nozzle onto a print bed to provide a 3D structure; and
(c) curing the 3D structure.

In a fifth aspect of the invention there is provided a method for preparing a scaffold for use in 3D tissue culture, the method comprising:
(a) providing a composition of the first aspect;
(b) placing the composition in a mould; and
(c) curing the composition.

Is a sixth aspect of the invention there is provided a 3D scaffold obtained or obtainable by the method of the fourth or fifth aspect.

In a seventh aspect of the invention there is provided a method for preparing a tissue, the method comprising:
(a) providing a scaffold of the second, third or sixth aspect, wherein the scaffold comprises mammalian cells;
(b) culturing the scaffold under physiological conditions.

In an eighth aspect of the invention there is provided a tissue obtained or obtainable by the method of the seventh aspect.

In a ninth aspect of the invention there is provided the scaffold of the second, third or sixth aspect, or the tissue of the eighth aspect, for use in a method of treatment.

In a tenth aspect of the invention there is provided the scaffold of the second, third or sixth aspect, or the tissue of the eighth aspect, for use in a method of tissue repair. The method of tissue repair typically comprises implanting scaffold or tissue into a diseased tissue or organ.

In an eleventh aspect of the invention there is provided an acellular medical implant comprising the scaffold of the second, third or sixth aspect, or the tissue of the eighth aspect.

In a twelfth aspect of the invention there is provided the use of the scaffold of the second, third or sixth aspect to promote vascularisation of endothelial cells.

These and other aspects and embodiments of the invention are described in further detail below.

### SUMMARY OF THE FIGURES

The present invention is described with reference to the figures listed below.

FIG. 1 is a schematic providing an overview of the PEGylation of fibronectin and the formation of a hydrogel. A is a schematic of the PEGylation protocol followed to functionalise fibronectin, where native fibronectin was denatured using TCEP and then PEGylated using Michael-type addition reaction with 4-arm-PEG-maleimide at a ratio 1:4 FN:PEG. After PEGylation, a step of alkylation followed using iodoacetamide to block unreacted thiol groups. The modular composition of fibronectin is shown at right: domain I (large ellipsis), domain II (the two small circles between domain I at left), and domain III (the remaining small circles), and cysteine residues are marked as *. B is a representation of fibronectin hydrogels formed through the reaction of full-length fibronectin (with exposed thiols), fibronectin PEGylation and additional dithiol or VPM crosslinker with 4-arm-PEG-Maleimide.

FIG. 2 is a schematic showing the components of a hydrogel according to an embodiment of the invention. A shows native fibronectin and GelMA. B shows fibronectin denatured and then PEGylated using Michael-type addition reaction with 4-arm-PEG-maleimide at a ratio 1:4 FN:PEG molecules. After PEGylation, a step of alkylation using iodoacetamide is typically used to block unreacted thiol groups (no shown). D is a representation of two scaffodls formed from FN/GelMa bioinks after curing. At left is a scaffold comprising native (non-PEGylated) fibronectin and GelMa, and at right is a scaffold comprising PEGylated fibronectin and GelMa. As shown, the fibronectin is not covalently bound into the hydrogel.

FIG. 3 shows the release of PEGylated or native fibronectin from different hydrogels. Native fibronectin in a PEG-maleimide hydrogel (top line), PEGylated fibronectin in a PEG-maleimide hydrogel (second line), native fibronectin in a GelMA hydrogel (central line), PEGylated fibronectin in a GelMA hydrogel (fourth line), and PEGylated fibronectin in a PEG-acrylate hydrogel (bottom line).

FIG. 4 shows the promotion of vascularisation in 3D scaffolds. A are brightfield images of HUVECs and HDF taken after 9 days in culture (scale bar: 50 μm). They hydrogels are: PEGylated fibronectin in GelMA (top left), PEGylated fibronectin in PEG-malaimide (top centre), native laminin in GelMA (top right), GelMA alone (bottom left), native fibronectin in GelMA (bottom centre), and PEGylated fibronectin in PEG-acrylate (bottom right). B is a bright field image of HUVECs sprouting in 3D FN PEGylated in GelMa after 7 days (scale bar: 50 μm).

### DETAILED DESCRIPTION OF THE INVENTION

The present invention provides a composition for use in 3D tissue culture. The composition comprises a biocompatible polymer suitable for the preparation of a hydrogel and a modified extracellular matrix (ECM) protein, or a fragment thereof. The modified extracellular matrix protein comprises a synthetic linker-L-which terminates in a blocking group-B that does not react with the biocompatible polymer during polymerisation. As such, the modified extracellular matrix protein is not covalently bound to the hydrogel. Instead, the modified extracellular matrix protein is entrapped within the hydrogel.

The composition of the invention typically comprises suitable rheological properties to allow it to be printed using 3D printing techniques. Accordingly, the composition may be referred to as a bioink.

### Biocompatible Polymer

The composition of the invention comprises a biocompatible polymer, i.e. a polymer that is not toxic or harmful to living cells or tissues. Thus, cells can be directly mixed into the polymerizable composition before the composition is polymerised to form a hydrogel comprising viable cells.

The biocompatible polymer can be polymerised (cured) to form the hydrogel. The biocompatible polymer may contain reactive groups that can be directly cross-linked to form the hydrogel, or it may contain groups that can be reacted with a separate cross-linker to form the hydrogel. Methods of polymerisation are discussed below.

The biocompatible polymer is suitable for preparation of a hydrogel. Hydrogels typically contain a network of cross-linked hydrophilic polymer chains and water. Thus, the biocompatible polymer is typically also hydrophilic.

Suitable biocompatible polymers include polymers based on proteins (e.g. polypeptides), polymers based on sugars (e.g. polysaccharides) and polymers based on synthetic motifs (synthetic polymers).

Examples of suitable polypeptides include collagen and gelatin. Derivatives of these polypeptides, such as methacryloyl derivatives, may also be used. Specific examples of suitable polypeptides include collagen methacryloyl (ColMA) and gelatin methacryloyl (GelMA).

Examples of suitable polysaccharides include cellulose (such as cellulose nanofibrils), hyaluronic acid, alginate, agar, pectin, chitosan, gellan gum, carrageenan and derivatives thereof. Specific examples of suitable polysaccharides include alginate and hyaluronic acid methacrylate (HA-MA).

Examples of suitable synthetic polymers include polyethylene glycol (PEG), polypropylene glycol (PPG), polytetramethylene glycol (PTMG), poly-3-hydroxybutyrate (P3HB), poly-4-hydroxybutyrate (P4HB), polyhydroxyvalerate (PHV), polyhydroxyhexanoate (PHH) and polyhydroxyoctanoate (PHO). Derivatives of these components, such as (meth)acrylate derivatives, may also be used. Specific examples of suitable synthetic polymers include (meth)acrylate terminated PEG.

In some embodiments, the biocompatible polymer is selected from GelMA, ColMA, alginate, HA-MA and (meth)acrylate-terminated PEG. Preferably, the biocompatible is selected from GelMA, alginate and (meth)acrylate-terminate PEG.

Preferably, the biocompatible polymer is a (meth)acryloyl terminated polymer. Typical examples of (meth)acryloyl terminated oligomers include (meth)acryloxyl terminated oligomers and (meth)acrylamidyl terminated oligomers. Accordingly, the biocompatible polymer is selected from GelMA, ColMA, HA-MA and (meth)acrylate terminated PEG. More preferably, the biocompatible polymer is selected from GelMA, HA-MA and (meth)acrylate terminated PEG. Even more preferably, the biocompatible polymer is selected from GelMA and HA-MA. Most preferably, the biocompatible polymer is GelMA.

In a preferred embodiment, the biocompatible polymer is gelatin methacryloyl (GelMA).

The number of methacryloyl groups per gelatin molecule is defined using the degree of substitution (DS; also known as degree of methacrylolyation, DM, units of %). The DS of the GelMA (DS_{GelMA}) may be known, or it may be determined using standard techniques such as ¹H-NMR spectroscopy or colorimetric methods (TNBS and Fe(III)-hydroxamic assays).

GelMA having any suitable DS may be used. Typically, the DS is from 40% to 100%. Preferably, the DS is from 40% to 80%, more preferably 40% to 60%, and most preferably 45% to 65%.

The strength of the GelMA is defined using the Bloom number (units of g). The Bloom number may be known, or it may be determined using standard techniques such as using a Bloom gelometer (typically measured at 10° C.). Bloom number is proportional to the average molecular mass of the GelMA (in Da).

GelMA having any suitable bloom number may be used. Typically, the bloom number is in from 50 to 325. Preferably, the bloom number is from 150 to 325.

The composition of the invention typically comprises 2 wt % to 20 wt % GelMA. Preferably, the composition of the invention comprises the GelMA in an amount of from 2 wt % to 15 wt %, more preferably 2 wt % to 10 wt %, even more preferably 5 wt % to 10 wt %.

GelMA from any source may be used. GelMA may be prepared from gelatin, such as Type B gelatin, by reaction with methacrylic anhydride.

In one embodiment, the biocompatible polymer is alginate, such as sodium alginate.

Alginate from any source may be used, such as alginate derived from seaweed (e.g. bown algae) including Laminaria hyperborea, Laminaria digitata, Laminaria japonica, Ascophyllum nodosum, and Macrocystis pyrifera (kelp).

Alginate is a polysaccharide comprising a-d-mannuronic acid (M residues) and β-I-guluronic acid (G residues). Typically, alginate is a linear copolymer comprising blocks of sequential M residues (MMMMMM), sequential G residues (GGGGGG) and alternating M/G residues (GMGMGM). The M and G contents of alginate depend on the extraction source. Only G-blocks are thought to interact with divalent cations (e.g., Ca²⁺) to form ionic crosslinks, and so hydrogels prepared from alginate with a high content of G residues exhibit higher stiffness than those with a low amount of G residues hydrogels. Accordingly, alginate with a high G-content is preferred. The composition of the invention typically comprises alginate with a G-block content of 10% to 70%. Preferably, the composition of the invention comprises alginate with a G-block content of 20% to 60%.

The G-content of the alginate may also be defined using the ratio of G-residues to M-residues (G/M). The composition of the invention typically comprises alginate with a G/M ratio of from 0.2 to 5.0. Preferably, the the composition of the invention comprises alginate with G/M ratio of from 0.5 to 5.0, more preferably from 1.0 to 5, and most preferably from 1.5 to 5.0.

The molecular weight (Mₙ) of the alginate is typically from 30,000 Da to 400,000 Da.

The composition of the invention typically comprises 1 wt % to 5 wt % alginate. Preferably, the composition of the invention comprises the alginate in an amount of from 2 wt % to 5 wt %, more preferably 2 wt % to 5 wt %.

In one embodiment, the biocompatible polymer is hyaluronic acid methacrylate (HA-MA).

The number of methacryloyl groups per hyaluronic acid molecule is defined using the degree of substitution (DS, defined as the number of methacrylate groups per 100 disaccharide units, in %). The DS of the HA-MA (DS_{HA-MA}) may be known, or it may be determined using standard techniques such as 1H-NMR spectroscopy.

HA-MA having any suitable DS may be used. Typically, the DS is from 10% to 60%. Preferably, the DS is from 10% to 50%, more preferably from, 10% to 30%, even more preferably from 15% to 25%.

The length of a polymer, such as HA-MA, is typically defined by the molecular weight of the polymer in Daltons (Da). More specifically, using the number average molecular weight (Mₙ). The molecular weight may be known, or it may be determined using standard techniques, such as size exclusion chromatography (SEC) or gel permeation chromatograph (GPC).

The molecular weight (Mₙ) of the HA-MA is typically in from 10 kDa to 1,000 kDa. Preferably, the molecular weight of the HA-MA is from 100 kDa to 1,000 Da, more preferably 250 kDa to 1000 kDa.

The composition of the invention typically comprises 1 wt % to 5 wt % HA-MA. Preferably, the composition of the invention comprises the HA-MA in an amount of from 2 wt % to 3 wt %.

In one embodiment, the biocompatible polymer is a (meth)acrylate terminated polyethylene glycol (PEG). That is, acrylate terminated polyethylene glycol (PEG-AC) or methacrylate terminated polyethylene glycol (PEG-MA). The (meth)acrylate terminated PEG may be branched or unbranched.

Examples of linear (unbranched) (meth)acrylate terminated PEGs include PEG dimethacrylate and PEG diacrylate.

Examples of branched (meth)acrylate terminated polyethylene glycol includes (meth)acrylate terminated multi-arm PEGs (star PEGs). The multi-arm PEG may be selected from 3-arm, 4-arm, 6-arm, 8-arm or Y-shaped PEGs.

Multi-arm PEGs may be defined by their core (branched) structure. 3-arm PEGs include those having a glycerol or a trimethylolpropane core. 4-arm PEGs include those having a pentaerythritol core. 6-arm PEGs include those having a dipentaerythritol core. 8-arm PEGs include those having a hexaglycerol or tripentaerythritol core. Y-shaped PEGs include those having a glycine or alanine core.

Preferably, the (meth)acrylate terminated PEG is selected from PEG dimethacrylate, PEG diacrylate and 4-arm PEG acrylate. More preferably, the (meth)acrylate terminated PEG is a 4-arm PEG acrylate.

The length of the (meth)acrylate terminated PEG is typically defined using the molecular weight (Mₙ) of the (meth)acrylate terminated PEG in Daltons (Da). The molecular weight may be known, or it may be determined using standard techniques.

The molecular weight (Mₙ) of the (meth)acrylate terminated PEG is typically from 100 Da to 50,000 Da. Preferably, the molecular weight of the linker is from 1,000 Da to 40,000 Da; more preferably 2,000 Da to 40,000 Da; even more preferably 5,000 Da to 40,000 Da; and most preferably 5,000 Da to 20,000 Da.

The composition of the invention typically comprises 2 wt % to 20 wt % (meth)acrylate terminated PEG. Preferably, the composition of the invention comprises the (meth)acrylate terminated PEG in an amount of from 2 wt % to 15 wt %, more preferably 2 wt % to 10 wt %, even more preferably 5 wt % to 10 wt %.

The composition of the invention may comprise a combination of biocompatible polymers, for example a combination of biocompatible polymers selected from GelMA, Alginate, HA-MA and PEG-AC. Typically, PEG-AC may be imported into the composition to increase the mechanical strength and the stability of the hydrogel.

The composition of the invention typically comprises a total amount of 1 wt % to 20 wt % of the biocompatible polymer or polymers. Preferably, the composition of the invention comprises the biocompatible polymer or polymers in total amount of 2 wt % to 20 wt %, more preferably 2 wt % to 10 wt %.

### Extracellular Matrix (ECM) Protein

The composition of the invention comprises a modified extracellular matrix (ECM) protein, or a fragment thereof. Extracellular matrix proteins play an important role in providing structure to the extracellular matrix, and can also influence cell growth and behaviour by binding and controlling the release of biological molecules such as growth factors (GFs).

The extracellular matrix protein may be selected from fibronectin, laminin, collagen or vitronectin.

Fibronectin (FN) is a high molecular weight glycoprotein dimer of two subunits (approximately 220 kDa each) linked by a disulfide bond near their carboxyl termini. Each subunit comprises three types of repeating units or “modules” (designated types I, II, and III) and a variable region. These repeats contain intermolecular binding sites that mediate interactions with other FN molecules (FNI₁₋₅, FNIII₁₋₂), other extracellular matrix (ECM) components, integrins (FNIII₉₋₁₀) and GFs (FNIII₁₂₋₁₄), and hence play roles in (for example) cell adhesion, migration, proliferation, and extracellular matrix assembly at a wound site. FN has two physical forms: a secreted soluble form in wound exudates and blood plasma, and a fibrillar form in the provisional extracellular matrix that is assembled by fibroblasts. Fibronectin binds a wide range of GFs, including bone morphogenetic protein 2 (BMP2, which drives bone formation) and vascular endothelial GF (VEGF, which stimulates angiogenesis). Preferably, the fibronectin is full-length fibronectin.

Laminins (LMs) are high molecular weight (approx. 400-900 kDa) multimeric glycoproteins, formed of three chains (α, β, γ) and located mainly in the basement membrane. Each of the α, β and γ chains have a number of genetic variants (5, 4 and 3 respectively in humans) and laminin molecules can therefore be named according to their chain composition. They have an important role in cell differentiation, proliferation, and migration. There are sixteen isoforms of LM having different chain compositions and with tissue-dependent distribution, including LM111, LM211, LM121, LM221, LM3A32, LM3B32, LM3A11, LM3A21, LM411, LM421, LM511, LM521, LM213, LM423, LM522 and LM523. LM332 is predominantly found in epithelial, bone and vascular tissues. LM411 and LM111 are found in the central and peripheral nervous system (CNS and PNS). LM221 and LM521 are mostly expressed in muscle and liver, respectively. Laminin isoforms bind many different GFs from the VEGF/PDGF, FGF, BMP, and NT families, in addition to HB-EGF and CXCL12γ. In some embodiments, the laminin is selected from LM521 and LM332.

Collagens are the main structural protein in the extracellular matrix. Typically three collagen polypeptide chains associate with one another in a triple helix to form a tropocollagen molecule, which can then assemble into higher-order structures such as fibrils and other aggregates. Each polypeptide chain comprises repeats of the triplet amino acid sequences Gly-X-Y, Gly-Pro-X and Gly-X-Hyp, where X and Y are any amino acid and Hyp is hydroxyproline. Overall, glycine, proline and hydroxyproline account for approximately half of the total amino acid content of collagen. Non-proline rich regions in particular may have roles in binding to (for example) cell surface molecules or other components of the ECM. At least 28 types of human collagen have been described, and any of them may find use in the compositions and methods described herein. Types I, II, IIII, IV and V may of be particular use. Type I collagen is the main collagen found in skin, tendon, vasculature, organs and bone. Type II collagen is the main collagen found in cartilage. Type IV collagen is located in basal lamina (basement membrane). Type V collagen is found on cell surfaces and in hair and placenta. In their higher order structures, collagens may be fibrillar or non-fibrillar. IN some embodiment, the collagen is Type I collagen.

Vitronectin is a 54 kDa glycoprotein (containing 458 amino acids) that exists in either a single chain form or a clipped form comprising two chains linked by a disulfide bond. It contains three primary domains, namely an N-terminal somatomedin B domain, plus a central domain and a C-terminal domain both of which have homology to hemopexin. Vitronectin contains an RGD sequence which acts as a binding site for membrane-bound integrins and serves to anchor cells to the extracellular matrix. Vitronectin can bind and sequester hepatocyte growth factor (HGF).

In some embodiments, the extracellular matrix protein is selected from fibronectin or laminin.

Extracellular matrix proteins from any suitable source may be used. Extracellular matrix proteins may be extracted from tissue. Alternatively, recombinant protein may be used. For example, recombinant human laminins may be produced in Hek293 cells or purified from mouse Engelbreth-Holm-Swarm (EHS) sarcoma (Doi et al., 2002). Human plasma fibronectin may be isolated and purified from human plasma. Fragments of the extracellular matrix protein may be produced recombinantly by bacterial or mammalian cells. The ECM proteins may be from the same organism as the cells to be incorporated into or grown on the resultant scaffold. In this context “from” is not intended to indicate any particular mode of expression or purification, but simply that the protein has the same sequence as the corresponding molecule(s) found in the organism from which the cells will be derived. It will be understood that they may have been expressed recombinantly in a different host organism.

Fragments of the extracellular matrix protein may also be modified and used in the composition of the invention. Fragments of extracellular matrix proteins typically retain the ability to bind and sequester growth factors. Thus, the extracellular matrix protein fragment typically contains at least one GF binding site. Additionally or alternatively, the extracellular matrix protein fragment may retain the ability to bind to a cell, e.g. to an adhesion molecule (such as an integrin) on the surface of a cell, e.g. on the type of cell to be incorporated into or onto the resultant scaffold. The fragment preferably contains an RGD sequence.

In one embodiment, a fragment of laminin is used. The laminin fragment may be a fragment of any laminin isoform, such as a fragment of LM332, LM411, or LM521. The laminin fragment may be or comprise a laminin-derived peptide, such as IKVAV or YIGSR. These sequences are known to promote cell adhesion. The laminin-derived fragment may contain a heparin-binding domain (HBD), which binds growth factors.

In one embodiment, a fragment of fibronectin is used. The fibronectin fragment may be or comprise a subunit (or module) selected from FNI₁₋₅, FNIII₁₋₂, FNIII₇, FNIII₉₋₁₀, FNIII₁₀, FNIII₁₂₋₁₄, and FNIII₁₂₋₁₅. Preferably, the fibronectin fragment comprises FNIII₉₋₁₀ and/or FNIII₁₂₋₁₄ (which bind growth factors). The fibronectin fragment may also be or comprise a fibronectin-derived peptide, such as GRGDSPC. This sequence contains RGD, which promotes cell adhesion.

In one embodiment a fragment of collagen is used. The collagen fragment may contain a binding motif selected from RGD and GFOGER.

Fragments of the extracellular matrix proteins will typically comprise at least 10, at least 25, at least 50, at least 100, at least 150 or at least 200 contiguous amino acids of the relevant full-length protein. They may comprise one or more complete domains of the relevant protein, typically with appropriate folding (i.e. secondary and tertiary structure).

The composition of the invention typically comprises 50 μg/ml to 1,000 μg/ml of the modified extracellular matrix protein, or fragment thereof.

The quantity of modified extracellular matrix protein, or a fragment thereof, can be adjusted to mimic different tissue environments as appropriate. For example, basement membrane and neural tissues typically contain higher levels of laminin, while connective tissues typically contain higher levels of fibronectin. The quantity of modified extracellular matrix protein, or a fragment thereof, can be adjusted to influence the growth and differentiation of cells in the scaffold.

The quantity of modified extracellular matrix protein, or a fragment thereof, is typically small. Therefore, it is typically given in μg/ml of composition. Typically, the composition comprises around 50 μg/ml to 1,000 μg/ml of the modified extracellular matrix protein or fragment thereof. Roughly, this equates to around 0.005 wt % to 0.1 wt %.

Typically, the composition of the invention comprises a modified laminin, or a modified fragment of laminin, in an amount of 50 μg/ml to 500 μg/ml. Preferably, the composition comprises a modified laminin, or a modified fragment of laminin, in an amount of 100 μg/ml to 500 μg/ml.

Typically, the composition of the invention comprises a modified fibronectin, or a modified fragment of fibronectin, in an amount of 100 μg/ml to 1,000 μg/ml. Preferably, the composition comprises a modified fibronectin, or a modified fragment of fibronectin, in an amount of 250 μg/ml to 750 μg/ml.

The extracellular matrix protein is modified with a group comprising a synthetic polymer. The synthetic polymer is attached to the extracellular matrix protein, for example by covalent attachment to the side chain of an amino acid within the extracellular matrix protein, or to the C- or N-terminal of the protein. The synthetic polymer enables the extracellular matrix protein to be uniformly distributed throughout a hydrogel formed from the composition of the invention.

The modified extracellular matrix proteins comprising a group:

-L-B
wherein -L- is a synthetic linker and —B is a blocking group.

The linker (-L-) of the modified extracellular matrix protein comprises a group for connection (i.e. covalent connection) of the extracellular matrix protein to the blocking group (—B). Typically, the linker comprises a divalent group in which the one of the free valencies forms part of a single bond to an amino acid residue in the extracellular matrix protein and the remaining free valency forms part of a single bond to the blocking group (—B).

Preferably, the linker is a stable linker. That is, the linker comprises a group that is not substantially cleaved or degraded in vivo. A stable linker is typically unreactive at physiological pH, and not substantially degraded by enzymatic action in vivo.

The linker does not contain functional groups that can react with the biocompatible polymer. As such, the extracellular matrix protein is not covalently bound into the hydrogel matrix via the linker. The linker may be described as unreactive or inert.

The linker comprises a synthetic polymer that suitable for preparation of a hydrogel. Thus, the linker comprises a hydrophilic polymer.

The linker typically comprises a polymer group selected from polyether and polyester groups.

Suitable polyether groups include polyalkylene gloycols, such as polyethylene glycol (PEG), polypropylene glycol (PPG) and polytetramethylene glycol (PTMG).

Suitable polyester groups include poly-3-hydroxybutyrate (P3HB), poly-4-hydroxybutyrate (P4HB), polyhydroxyvalerate (PHV), polyhydroxyhexanoate (PHH) and polyhydroxyoctanoate (PHO).

Preferably, the linker comprises a polyether group. More preferably, the linker comprises a polyethylene glycol group.

The linker may be linear or branched.

Suitable branched polyether groups include multi-arm PEGs (star PEGs) and dendritic PEGs. The multi-arm PEG may be selected from 3-arm, 4-arm, 6-arm, 8-arm and Y-shaped PEGs.

Preferably, the linker comprises a polymer selected from linear, 3-arm or 4-arm PEG. More preferably, the linker comprises a 4-arm PEG.

The linker may be attached to the extracellular matrix protein using any suitable bioconjugation method. A preferred method is to use a sulfhydryl-reactive cross-linker such as a maleimide, which selectively reacts with sulfhydryl groups present in the extracellular matrix molecule, such as in a cysteine side chain, to form a stable thioether linkage.

In such cases, the linker -L- has the formula (I):

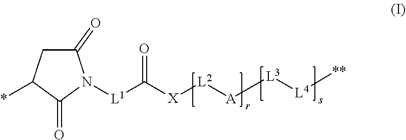

where:
   L¹ is C₁₋₆ alkylene;
   X is O or NH;
   L² and L³ are independently polyether or polyester groups;
   A is a branching group;
   L⁴ is selected from a covalent bond, a C₁₋₆ alkylene group or a group:

-L^{4A}-L^{4X}-C(O)-L^{4B-}
where L^{4A} and L^{4B} are independently C₁₋₆ alkylene groups, and L^{4X} is O or NH;
r is 0 or 1;
s is from 1 to 8;
* is the attachment point with the ECM protein; and
** is the attachment point with the blocking group (—B).

The C₁₋₆ alkylene group may be linear or branched. Examples of suitable linear C₁₋₆ alkylene groups include methylene (methanediyl), ethylene (ethane-1,2-diyl), propylene (propane-1,3-diyl), butylene (butan-1,4-diyl), pentylene (pentan-1,5-diyl) and hexylene (hexan-1,6-diyl). Examples of suitable branched C₁₋₆ alkylene groups include ethane-1,1-diyl and propane-1,2-diyl.

Preferably, L¹ is C₁₋₄ alkylene. More preferably L¹ is ethylene.

Preferably X is NH.

Suitable polyether groups include PEG (—CH₂CH₂O—)ₙ, PPG (—CH₂CH₂CH₂O—)ₙ and PTMG (—CH₂CH₂CH₂CH₂O—)ₙ. Suitable polyester groups include P3HB, P4HB, PHV, PHH and PHO.

Preferably L² and L³ are independently polyether groups. More preferably, L² and L³ are independently PEG groups.

Preferably L⁴ is a group:

-L^{4A}-L^{4A}-C(O)-L^{4B}-
where L^{4A} and L^{4B} are independently C₁₋₆ alkylene groups, and L^{4X} is O or NH.

Preferably L^{4A} and L^{4B} are independently C₁₋₄ alkylene. More preferably L^{4A} and L^{4B} are independently ethylene.

### Preferably L^{4X} is NH.

In one embodiment, r is 0. In such cases, the linker -L- is linear (unbranched). The linker may be represented by the formula (II):

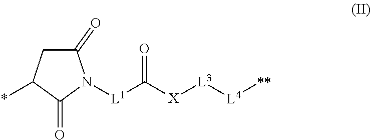

where L¹, X, L³, L⁴, * and ** are as described for formula (I), and the same preferences apply.

In a preferred embodiment, r is 1. In such cases, the linker comprises branching group A, and the linker -L- may be described as a branched linker.

Suitable branching groups A may be based on glycerol, trimethylolpropane, pentaerythritol, dipentaerythritol, tripentaerythritol, hexaglycerol, glycine and alanine. Examples of suitable branching groups A are set out below:

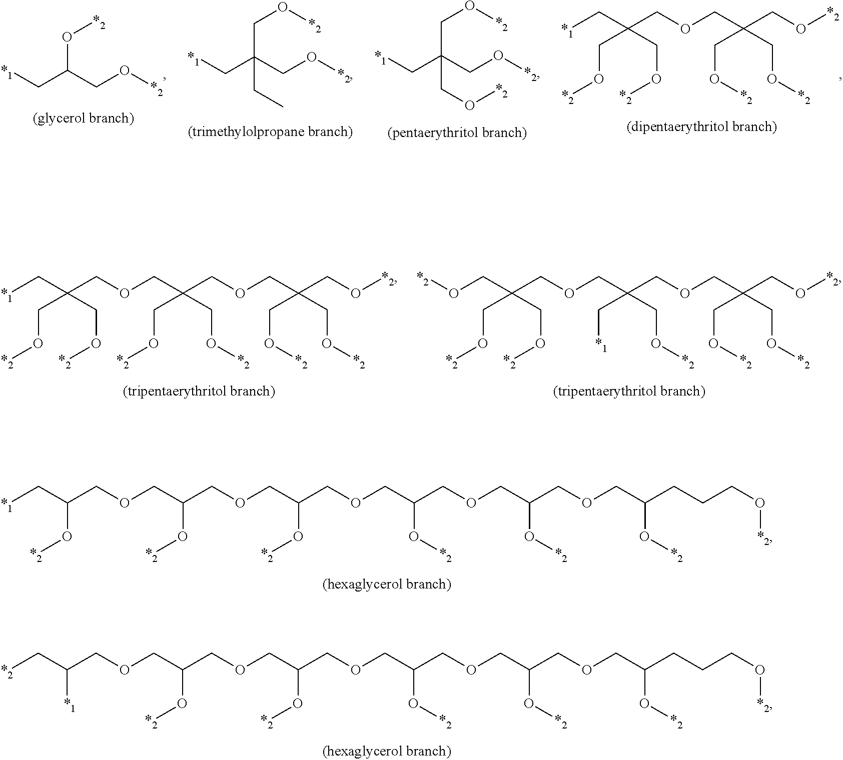

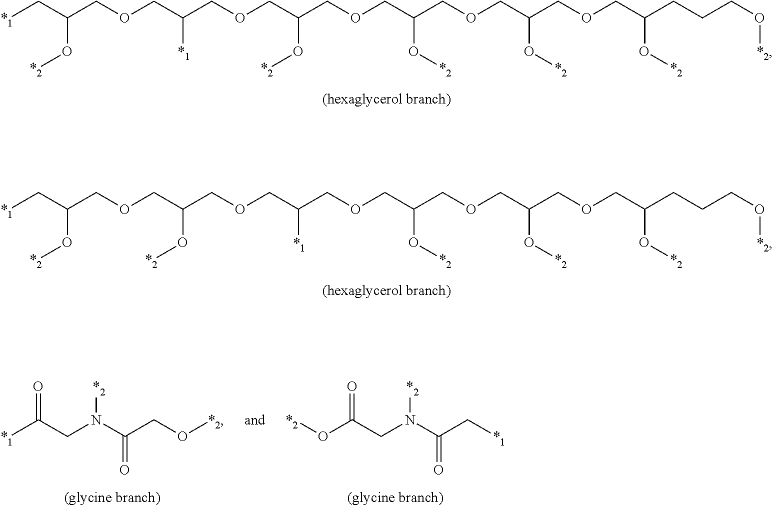

where *¹ is the attachment point with the linker unit L², and *² is the attachment point with the linker unit L³.

In a particularly preferred embodiment, the branching group A is based on pentaerythritol. In such cases, the linker may be represented by the formula (III):

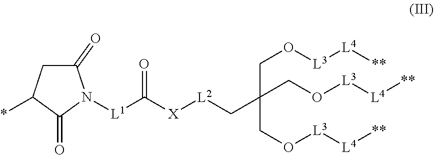

where L¹, L², L³, L⁴, X, * and ** are as described for formula (I), and the same preferences apply.

In the worked examples, a 4-arm PEG maleimide is used. In such cases, the linker may be represented by the formula (IV):

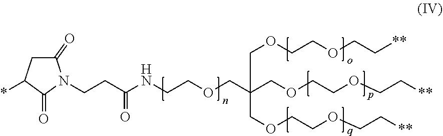

where * is the attachment point with the ECM protein and ** is the attachment point with the blocking group (—B).

The groups n, o, p and q represent the number of repeats in the PEG chain. The sum of the groups n, o, p and q is set to provide a linker having a given molecular weight.

The length of the linker is typically defined by the molecular weight of the linker in Daltons (Da). More specifically, using the number average molecular weight (Mₙ). The molecular weight may be known, or it may be determined using standard techniques, such as size exclusion chromatography (SEC) or gel permeation chromatograph (GPC).

The molecular weight (Mₙ) of the linker is typically from 100 Da to 50,000 Da. Preferably, the molecular weight of the linker is from 1,000 Da to 40,000 Da; more preferably 2,000 Da to 40,000 Da; even more preferably 5,000 Da to 40,000 Da; and most preferably 5,000 Da to 20,000 Da.

The blocking group (—B) of the modified extracellular matrix protein comprises a group which does not react with the biocompatible polymer during polymerisation. That is, the blocking group does not form a covalent bond with the biocompatible polymer under typical polymerisation conditions (see below). Thus, the modified extracellular matrix protein is not covalently bound to the hydrogel after polymerisation. Instead, the modified extracellular matrix protein is entrapped within the hydrogel.

Typically, the blocking group (—B) comprises a monovalent group in which the free valences forms part of a single bond to the linker group (-L-). The blocking group (—B) may be described as the terminal group of the synthetic polymer.

The blocking group is typically selected from hydrogen, C₁₋₆ alkyl, carboxylic acid, hydroxy, amine and maleimide.

Hydrogen blocking groups have the formula (—H). Suitable groups-L-B in which the blocking group (—B) is a hydrogen include unmodified polyethers and polyesters.

Suitable C₁₋₆ alkyl blocking groups include C₁₋₆ linear alkyl groups and C₁₋₆ branched alkyl groups. Examples of C₁₋₆ linear alkyl groups include methyl (-Me), ethyl (-Et), n-propyl (-nPr), n-butyl (-nBu), n-pentyl (-Amyl) and n-hexyl. Examples of C₁₋₆ branched alkyl groups include iso-propyl (-iPr), iso-butyl (-iBu), sec-butyl (-sBu), tert-butyl (-tBu), iso-pentyl, sec-pentyl, tert-pentyl, neo-pentyl, iso-hexyl, sec-hexyl, tert-hexyl and neo-hexyl.

Carboxylic acids blocking groups contain a terminal carboxylic acid group (—CO₂H). Examples of suitable carboxylic acid groups include C₁₋₆ carboxylic acid blocking groups such as —C(═O)(CH₂)₃CO₂H, —H₂CO₂H and —CO₂H. Examples of suitable groups -L-B in which the blocking group —B is a C₁₋₆ carboxylic acid group include PEG derivatised with glutaric acid or glycolic acid.

Amine blocking groups contain a terminal amine group (—NH₂). Examples of groups-L-B in which the blocking group-B is amine include PEG derivatised with 2-aminoethanol (ETA).

Maleimide blocking groups contain a terminal maleimide group:

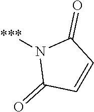

where, *** represents the attachment point with the linker group.

Examples of groups -L-B in which the blocking group —B is a maleimide include 4-arm PEG maleimide.

Preferably, the blocking group is selected from C₁₋₆ alkyl, carboxylic acid, alkylamine and maleimide. More preferably, the blocking group is maleimide. In such cases, the synthetic polymer may be described as a maleimide-terminated synthetic polymer (e.g. a maleimide-terminated PEG).

The number of groups -L-B per extracellular matrix protein may be defined using a degree of substitution (DS_{ECM}). The number of groups per extracellular matrix may be determined using standard techniques, such as by measuring the number of free thiol or amino groups before and after derivatisation. Standard assays for measuring thiol and amino contented are available, such as colorimetric or fluorometric assays including the 2,4,6-Trinitrobenzene Sulfonic Acid (TNBSA) assay. Mass spectrometry may also be used.

The composition of the invention typically comprises a population of extracellular matrix proteins, or extracellular matrix protein fragments. Typically, at least 50% of the extracellular matrix proteins or extracellular matrix protein fragments within the population are modified such that they contain a group -L-B. Preferably, at least 60% of the extracellular matrix proteins in the population are modified, more preferably at least 70% of the extracellular matrix proteins in the population are modified, even more preferably at least 80% of the extracellular matrix proteins in the population are modified, and most preferably at least 90% of the extracellular matrix proteins in the population are modified.

The modified extracellular matrix protein or protein fragment does not contain functional groups that can react with the biocompatible polymer to form a covalent bond, such as under the conditions under which the 3D scaffold is made and/or used. For example, the extracellular matrix protein or protein fragment does not contain functional groups that can react with (meth)acryloyl groups to form a covalent bond under the conditions in which the 3D scaffold is made and/or used. As such, the modified extracellular matrix protein is not covalently bound into the hydrogel matrix via components of the native extracellular matrix protein or protein fragment. Typical conditions for making and using the 3D scaffold are set out below (see, Scaffold Preparation and Tissue Preparation).

After modification of the extracellular matrix protein or protein fragment with the synthetic polymer, reactive functional groups remaining in the extracellular matrix protein or protein fragment may be blocked. For example, unreacted cystine residues may be blocked by, for example, alkylation. Any suitable alkylation reagent may be used, such as iodoacetamide.

In a particularly preferred embodiment, the extracellular matrix protein is modified with a 4-arm PEG maleimide having a Mₙ of 20 kDa.

### Additional Components

The composition of the invention typically comprises water. Commonly, the balance of the composition is water or a buffer solution. Typically, from 90 wt % to 98 wt % of the composition is buffer solution.

Any suitable buffered solution may be used. Examples of suitable buffer solutions include PBS (phosphate-buffered saline), TAPS, Bicine, Tris, Tricine, TAPSO, HEPES, TES, MOPS, PIPES, Cacodylate and MES buffers. Preferable, PBS buffer is used.

The composition of the invention may include a cross-linker. The additional cross-linkers may be used to adjust the viscosity and rate of biodegradation of the composition.

Suitable cross-linkers include thiolated cross-linkers. Examples of suitable thiolated cross-linkers include PEG dithiol, such as PEG diothiol 2 kDa, and protease-degradable peptides, such as VMP peptide (GCRDVPMSMRGGDRCG).

Where thiolate cross-linkers are used, modified extracellular matrix proteins having a maleimide blocking group should not be used, to avoid cross-linking the extracellular matrix protein into the hydrogel via the cross-linker. Non-thiol-reactive bocking groups such as C₁₋₆ linear alkyl groups should be used.

The amount of cross-linker is typically defined by reference to the molar amount of biocompatible polymer used in the composition. The crosslinker is typically present in a molar ratio of from 5:1 to 1:5 (biocompatible polymer: cross-linker). Preferably, the cross-linker is present in a molar ratio of 3:1 to 1:3 (biocompatible polymer: cross-linker), more preferably 2:1 to 1:2.

The composition of the invention may include an initiator to initiate polymerisation of the composition. Typically, the composition comprises a photoinitiator, which can initiate polymerisation on exposure to light of a given wavelength and intensity.

Any suitable photoinitiators may be included in the composition. Examples of suitable photoinitiators include from Irgacure 2959, LAP, VA-086 and eosine Y.

Preferably, the composition comprises a photoinitiator, more preferably LAP or Irgacure 2959.

The photoinitiator is typically present in an amount of 0.05 wt % to 0.50 wt %. Preferably, the photoinitiator is present in an amount of 0.1 wt % to 0.25 wt %.

The composition of the invention may also comprise growth factors or cytokines to influence cell growth and behaviour, such as proliferation and differentiation. The growth factors or cytokines can be bound and sequestered by the modified extracellular matrix proteins, or protein fragments, in the composition controlling their release.

The composition of the invention may, for example, comprise growth factors selected from bone morphogenetic proteins (BMPs), vascular endothelial growth factors (VEGFs), placental growth factors (PGFs), platelet-derived growth factors (PDGFs), fibroblast growth factors (FGFs), transforming growth factors (TGFs), hepatocyte growth factor (HGF), neurotrophins and hepatocyte growth factors (HGFs).

Specific examples of suitable BMPs include BMP-2, BMP-3 and BMP-7. Specific examples of suitable VEGFs include VEGF-A165, VEGF-B and EG-VEGF.

Specific examples of suitable PGFs include PGF-1, PGF-2 and PGF-3. Specific examples of suitable PDGFs include PDGF-AA, PDGF-AB, PDGF-BB and PDGF-DD. Specific examples of suitable FGFs include FGF-5, FGF-7, FGF-8, FGF-10, FGF-18 and FGF-21. Specific examples of suitable TGFs include TGFB1. Specific examples of suitable neurotrophins include BDNF and BNGF. Specific examples of suitable cytokines include CXCL-12y.

The quantity of growth factor or cytokine can also be adjusted to mimic different tissue environments as appropriate. Typically, each growth factor or cytokine is present in an amount of 500 ng/μl to 100 μg/μl.

The composition of the invention may comprise cells. The component parts of the composition, such as the biocompatible polymer, are not toxic or harmful to living cells or tissues and so cells can be directly mixed into the composition before the composition is polymerised. This provides an efficient method of forming a hydrogel comprising viable cells that are distributed uniformly throughout the hydrogel.

Any suitable cells may be incorporated into the composition. The cells may be prokaryotic (e.g. bacterial cells) or eukaryotic. Eukaryotic cells will typically be from multicellular organisms, including insects. However, mammalian cells will typically be preferred.

The cells may be from any suitable mammalian species, including rodents (e.g. mice, rats), lagomorphs (e.g. rabbits), felines (e.g. cats), canines (e.g. dogs), equines (e.g. horses), bovines (e.g. cows), caprines (e.g. goats), ovines (e.g. sheep), other domestic, livestock or laboratory animals, or primates (e.g. Old World monkey, New World monkey, apes or humans). Human cells are of particular use.

Suitable mammalian cells include neural cells, cardiomyocytes such as induced pluripotent stem cell-derived (iPSC-derived) cardiomyocytes, hepatocytes such as iPSC-derived hepatocytes, human umbilical vein endothelial cells (HUVECs), human dermal fibroblasts (HDFs), bone cells such as osteoblasts, osteoclasts and/or osteocytes, and stem cells such as human mesenchymal stem cells (hMSCs) and hematopoietic stem cells (HSCs).

The cells will typically be of the same species as a subject to whom (or to which) the scaffold or tissue of the invention is intended to be delivered. The cells may be autologous to the subject, e.g. they may have been obtained from the subject.

Suitable bacterial cells include lactic acid bacteria (such as Lactococcus lactis), Escherichia coli, and Bacillus subtilis.

The quantity of cells in the composition may be adjusted as appropriate. Typically, the composition comprises the cells in an amount of from 1×10⁴ to 1×10⁸ per ml, such as about 1×10⁶ per ml.

The composition of the invention may comprise thickening agents to allow precise control of viscosity. Any suitable thickening agent may be used. Suitable thickening agents include xanthan gum, gellan gum, nanocellulose, glucomannan, and pectin. Typically, the composition comprises the thickening agents in an amount of from 0.1 wt % to 5 wt %. More preferably, the composition comprises the thickening agents in an amount of from 0.5 wt % to 3 wt %.

The composition of the invention may comprise an osmotic diuretic to allow precise control of the osmotic pressure of the composition. Any suitable osmotic diuretic may be used. Suitable osmotic diuretics include mannitol and isosorbide.

The composition of the invention preferably has a viscosity from 1 to 1500 Pa·s⁻¹ at 25° C., preferably from 20 to 1500 Pa·s⁻¹ at 25° C.

The viscosity of the composition may be determined using standard techniques, such as using a rotational viscometer or a rheometer. Typically, the viscosity is measured at 25° C. and at a shear rate from 0 to 1,000 s⁻¹. Sample hydration should be maintained by addition of aqueous components (e.g. PBS buffer) at the exposed portions (e.g. sides) of the sample.

The viscosity of the composition may be adjusted by altering the concentration and molecular weight of the biocompatible polymer, as appropriate.

In a particularly preferred embodiment, the composition comprises:
(a) a (meth)acryloyl terminated polymer, such as GelMA; and
(b) a modified extracellular matrix (ECM) protein, or a fragment thereof, comprising a group:

-L-B

wherein:
the modified ECM protein is laminin or fibronectin;
-L- comprises a polyethylene glycol group; and
—B is a maleimide group.

In another particularly preferred embodiment, the composition comprises:
(a) a (meth)acryloyl terminated polymer, such as GelMA; and
(b) laminin or fibronectin, or a fragment of laminin or fibronectin, modified with a polyethylene glycol (PEG), such as a maleimide-terminated PEG.

Suitable polymers, proteins, protein fragments, PEG groups and additional components are set out above, and the same preferences apply.

In a particularly preferred embodiment, the composition comprises:
(a) 2 to 10 wt % (meth)acryloyl terminated polymer, such as GelMA; and
(b) 50 to 1,000 μg/ml laminin or fibronectin, or a fragment of laminin or fibronectin, modified with a polyethylene glycol (PEG), such as a maleimide-terminated PEG;
(c) 0 to 1 wt % photoinitiator;
(d) 90 to 98 wt % buffer solution;
(e) optionally, mammalian cells, such as HFDs and/or HUVECs.

Suitable polymers, proteins, protein fragments, PEG groups, buffer solutions, cells and additional components are set out above, and the same preferences apply.

### Scaffolds

The present invention also provides a 3D scaffold for use in tissue culture. The 3D scaffold may be obtained or obtainable by polymerisation of the polymerizable composition of the invention.

The 3D scaffold of the invention comprises:
(a) a hydrogel obtained or obtainable by polymerisation of a biocompatible polymer selected from polypeptides, polysaccharides and synthetic polymers; and
(b) a modified extracellular matrix (ECM) protein, or a fragment thereof, comprising a group:

-L-B
wherein -L- is a linker and —B is a blocking group.

The blocking group does not react with the biocompatible polymer during polymerisation, and so the modified extracellular matrix protein is not covalently bound to the hydrogel. Instead, the modified extracellular matrix protein is entrapped within the hydrogel.

Suitable biocompatible polymers, modified extracellular matrix proteins, linkers and blocking groups are set out above, along with suitable quantities for these components, and the same preferences apply.

The 3D scaffold comprises a hydrogel obtained or obtainable by polymerisation of a biocompatible polymer selected from polypeptides, polysaccharides and synthetic polymers. As such, hydrogels may be described as polypeptide-, polysaccharide-, or synthetic polymer-based hydrogels.

The hydrogel comprises a highly hydrated network of cross-linked polymer chains. Thus, the hydrogel comprises water. Typically, hydrogel comprises a buffer solution.

The 3D scaffold may contain further components such as cross-linkers, photoinitiators, growth factors and cells. Suitable buffers, cross-linkers, photoinitiators, growth factors and cells are set out above, and the same preferences apply.

The 3D scaffold may be in any suitable shape. Examples of suitable scaffold shapes include grids, such as multi-layered grid, single-layered sheets or filaments, and droplets. Solids of controlled volume are also useful, for example, for dosing into microwell plates for disease modelling or drug testing applications.

The 3D scaffold may have an elastic modulus (Young's modulus) of 1 to 40 kPa. The elasticity of the 3D scaffold can also be adjusted to mimic encourage the growth of different tissues as appropriate. For example, a low elastic modulus (approx. 1 kPa) may be used for the growth of neural and bone marrow tissues typically, an elastic modulus of approx. 5 kPa may be used for the growth of adipose tissues, approx. 10 kPa may be used for the growth of muscle tissue, while a 3D scaffold having high elastic modulus (approx. 30 kPa) may be used for the growth of osteoid (bone) tissue.

Typically, the 3D scaffold has an elastic modulus (Young's modulus) of 1 kPa to 15 kPa. Preferably, the elastic modulus is from 2 kPa to 10 kPa, more preferably 2 kPa to 8 kPa.

The elasticity of the 3D scaffold may be determined using standard techniques such as using a rheometer. Typically, strain sweeps are performed in the range of 0.01% to 1% at an angular frequency of 10 rad·s⁻¹ at different compression levels. Sample hydration should be maintained by addition of aqueous components (e.g. PBS buffer) at the exposed portions (e.g. sides) of the sample.

Alternatively, the elasticity of the 3D scaffold may be determined by nanoindentation using atomic force microscopy in force spectroscopy mode. Here, an indentation tip of known geometry (known surface area) is pressed into the material and a plot of load against penetration depth is recorded from which the mechanical properties of the material can be determined using, e.g., the Hertz model (for a spherical indenter).

In a particularly preferred embodiment, the 3D scaffold comprises:
(a) a hydrogel based on a (meth)acryloyl terminated polymer, such as a GelMA-based hydrogel; and
(b) a modified extracellular matrix (ECM) protein, or a fragment thereof, comprising a group:

-L-B

wherein:
the modified ECM protein is laminin or fibronectin;
-L- comprises a polyethylene glycol group; and
—B is a maleimide group.

In another particularly preferred embodiment, the 3D scaffold comprises:
(a) a hydrogel based on a (meth)acryloyl terminated polymer, such as a GelMA-based hydrogel; and
(b) laminin or fibronectin, or a fragment of laminin or fibronectin, modified with a polyethylene glycol (PEG), such as a maleimide-terminated PEG.

Suitable polymers, proteins, protein fragments, PEG groups and additional components are set out above, and the same preferences apply.

In a particularly preferred embodiment, the 3D scaffold comprises:
(a) a hydrogel based on a (meth)acryloyl terminated polymer, such as GelMA; and
(b) laminin or fibronectin, or a fragment of laminin or fibronectin, modified with a polyethylene glycol (PEG), such as a maleimide-terminated PEG;
(c) buffer solution;
(d) optionally, mammalian cells, such as HFDs and/or HUVECs.

Suitable polymers, proteins, protein fragments, PEG groups, buffer solutions, cells and additional components are set out above, and the same preferences apply

### Scaffold Preparation

The present invention also provides methods for preparing a scaffold for use in 3D tissue culture.

The method for preparing a scaffold may use 3D-printing techniques. In such cases, the method may comprise:
(a) providing a composition of the invention, as set out above;
(b) extruding the composition through a print nozzle onto a print bed to provide a 3D structure; and
(c) curing the 3D structure.

The use of 3D printing techniques to prepare scaffolds for use in 3D tissue culture is commonly known as 3D bioprinting.

Suitable 3D bioprinters are available and include, for example, 3D Discovery bioprinters (RegenHu, Switzerland), BIO X™ 3D Bioprinters (Cellink, Sweden), INKREDIBLE™ 3D Bioprinters (Cellink, Sweden), BIO MDX™ 3D Bioprinters (Cellink, Sweden) and 3D Bioplotter (EnvisionTEC).

The bioprinting method comprises extruding the composition through a print nozzle onto a print bed to provide a 3D structure. This may be referred to as the printing step, step (b).

During the printing step, the temperature of the print nozzle is set to provide suitable viscosity at a certain shear rate to the composition, such as low viscosity at extrusion pressure and high viscosity upon extrusion. Typically, the temperature of the print nozzle is 5° C. to 37° C.

During the printing step, the temperature of the print bed is set to allow for fixation of the composition on the print bed. Typically, the temperature of the print bed is 5° C. to 37° C., more commonly 10° C. to 20° C.

During the printing step, excessive pressures are avoided in order to mitigate damage to the composition. Typically, the pressure during the extrusion is 5 kPa to 200 kPa. Where cells are present in the composition, the lower pressures are used, and typical pressures are 5 kPa to 70 kPa, more commonly 10 kPa to 40 kPa. In the absence of cells, the full pressure range may be used, which and higher pressures are preferred for increased printing speed.

During the printing step, the nozzle diameter is typically 200 μm to 600 μm.

During the printing step, the horizontal speed of the nozzle is typically 2 mm/s to 20 mm/s, preferably 2 mm/s to 10 mm/s, and more preferably 2 mm/s to 8 mm/s.

Optionally, a template 3D printing technique is used. In temple printing, a template ink (a templating composition) is also printed during the printing process. This provides a 3D structure comprising both the templating ink and the bioink. In such cases, the printing step also comprises:
(b1) extruding a templating composition through a print nozzle onto the print bed to provide a 3D structure comprising the composition of the invention and the templating composition.

Suitable templating compositions include gelatin-based compositions. Typically, gelatin-based templating inks comprise 2 wt % to 10 wt % gelatin, preferably 3 wt % to 10 wt % gelatin, more preferably 5 wt % to 10 wt % gelatin. Another suitable templating composition is Pluronic F-127 (for example, Pluronics 40% by CELLINK).

The templating compositions typically comprises buffer solution. Commonly, the balance of the composition is water or a buffer solution. Any suitable buffered solution may be used. Examples of suitable buffer solutions include PBS (phosphate-buffered saline), TAPS, Bicine, Tris, Tricine, TAPSO, HEPES, TES, MOPS, PIPES, Cacodylate and MES buffers.

The templating composition may be printed at the same time as the composition of the invention using, for example, a multi-head 3D bioprinter.

Optionally, the templating ink can then be removed, for example by dissolution, to provide a 3D structure comprising cavities. In such cases, the method comprises, after step (c):
(d) liquifying and removing the cured templating composition.

The templating ink may be removed by heating. For example, gelatin may be removed by heating to 35° C. to 40° C., such as 37° C.

The method for preparing a scaffold may alternatively use a mould. In such cases, the method may comprise:
(a) providing a composition of the invention, as set out above;
(b) placing the composition in a mould; and
(c) curing the 3D composition.

Using a mould aids in the printing of bioinks having low viscosities.

Any suitable moulds may be used. Examples of suitable mould include those made from, or coated with, polydimethylsiloxane (PDMS).

Alternatively, a printing bath may be used in place of the mould. A printing bath is typically made of a sacrificial material that can be removed ager printing. Suitable materials include gelatin-based compositions (e.g. LifeSupport printing bath, Cellink, Sweden).

The methods for preparing a scaffold of the invention comprise a curing step, step (c), in which the composition of the invention is cured (polymerised) to form a 3D structure.

Any suitable curing method may be used, and suitable methods include thermal or photo curing. Preferably, photocuring is used. In such cases, the method comprises irradiating the composition or 3D structure with light, such as UV or visible light.

During the curing step, the wavelength of the light is typically from 315 nm to 410 nm.

During the curing step, the power of the light is typically from 2 mW/cm² to 50 mW/cm².

During the curing step, the light is typically applied for 5 s to 300 s.

The present invention also provides a 3D scaffold obtained or obtainable by the method of preparing a scaffold.

### Tissue Preparation

The present invention also provides methods for preparing a tissue for use in 3D tissue culture.

The method for preparing a tissue may comprise providing a scaffold of the invention, wherein the scaffold comprises mammalian cells. A scaffold comprising cells may be referred to a “seeded scaffold”.

The seeded scaffold may be obtained by polymerisation of a composition of the invention that contains mammalian cells. For example, the seeded scaffold may be obtained by printing a composition containing cells using the method set out above, or by using a mould as set out above.

Alternatively, the seeded scaffold may be obtained by introducing cells to a scaffold that does not contain cells. A scaffold that does not contain cells may be referred to as an “acellular scaffold”.

The acellular (bare) scaffold may be obtained by polymerisation of a composition of the invention that does not contain cells. For example, the acellular scaffold may be obtained by printing a composition that does not contain cells using the method set out above, or by using a mould as set out above. The step of introducing cells into the acellular scaffold may be described as a seeding step. The seeding step typically comprises contacting a seeding solution comprising cells with the acellular scaffold. The seeding solution is typically aqueous, and optionally comprises further components such as buffer components.

The method for preparing a tissue may comprise a step of culturing the scaffold, e.g. under physiological conditions. This may be referred to as the culturing step.

Typically, the temperature during the culturing step is from 20° C. to 40° C. Preferably, the temperature during the culturing step is from 25° C. to 40° C., more preferably 30° C. to 40° C., even more preferably 35° C. to 39° C. and most preferably about 37° C.

Typically, the pH during the culturing step is from 6 to 8. Preferably, the pH during the culturing step is about 7.

Typically, the osmolarity during the culturing step is from 275 to 300 mOsm/kg. Preferably the osmolarity during the culturing step is about 285 mOsm/kg.

The present invention also provides a tissue obtained or obtainable by the method for preparing a tissue.

### Methods of Treatment

The scaffolds and tissues of the invention may be useful in therapy. Accordingly, the present invention provides a method of treatment comprising delivering a scaffold or tissue of the invention to a subject in need thereof. The subject may be a human or animal, but is typically a human. Similarly, the present invention also provides a scaffold or tissue of the invention for use in a method of treatment. The method of treatment may be a method of treatment of a human or animal, but is typically treatment of a human.

The scaffolds and tissues of the invention may be useful for replacing or supplementing damaged tissue. Accordingly, the present invention also provides a method of tissue repair comprising implanting a scaffold or tissue into a subject in need thereof, e.g. into or onto a damaged or diseased tissue or organ. Similarly, the present invention also provides a scaffold or tissue of the invention for use in a method of tissue repair, the method comprising implanting the scaffold or tissue into a subject in need thereof, e.g. into or onto a damaged or diseased tissue or organ. The damaged or diseased tissue or organ may be a human or animal tissue or organ, but is typically a human tissue or organ.

The tissue for repair using the scaffolds and tissues of the invention may be, for example, osseous tissue (bone), cartilaginous tissue, muscle tissue, cardiac tissue, hepatic tissue and marrow adipose tissue. The tissue for repair may also be lung, pancreatic, kidney, muscle, neural, skin, retinal, adipose, cancer, or connective tissues (cartilage, tendon, ligament).

As noted above, where the scaffold or tissue contains cells, those cells will typically be of the same species as the subject. For example, the cells may be autologous to the subject. The cells may have been obtained from the subject. The methods of the invention may comprise a step of providing a sample of cells obtained from the subject. They may (or may not) comprise the step of obtaining the cells from said subject. The cells may optionally be cultured in vitro, before being contacted with a polymerisable composition or acellular scaffold of the invention.

### Medical Devices

The scaffolds and tissues of the invention may also be useful in medical devices, for example as acellular implants. Accordingly, the present invention also provides an acellular implant comprising a scaffold or tissue of the invention. The acellular implant of the invention maybe useful for replacing or supplementing damaged osseous tissue (bone), cartilaginous tissue, muscle tissue, cardiac tissue, hepatic tissue, marrow adipose tissue and connective tissue.

### Tissue Models

The scaffolds and tissues of the invention may also be useful in tissue models for drug, allergen, and cosmetic testing. Accordingly, the present invention also provides a method of assessing a drug, allergen or cosmetic, the method comprising:
i) providing a tissue model comprising a scaffold or tissue of the invention; and
ii) contacting the tissue model with a drug, allergen or cosmetic.

### Uses

The scaffolds of the present invention hold provides excellent retention of the modified extracellular matrix proteins, which are uniformly distributed throughout the scaffold and retain their biological function. Thus, they can act as a reservoir for growth factors. Accordingly, the scaffolds of the invention may promote vascularisation of endothelial cells, formation of bone, or formation of other tissue structures, particularly two or three dimensional tissue structures.

Accordingly, the present invention also provides the use of a scaffold of the invention to promote vascularisation of endothelial cells or formation of bone.

### Kits

The present invention also provides a kit comprising:
(a) a biocompatible polymer suitable for the preparation of a hydrogel selected from polypeptides, polysaccharides and synthetic polymers; and
(b) a modified extracellular matrix (ECM) protein, or a fragment thereof, comprising a group:

-L-B

wherein -L- is a synthetic linker and —B is a blocking group.

Suitable biocompatible polymers, modified ECM proteins, linkers and blocking groups are set out above, and the same preferences apply.

The kit may contain further components such as buffers, cross-linkers, photoinitiators, growth factors and cells. Suitable buffers, cross-linkers, photoinitiators, growth factors and cells are set out above, and the same preferences apply.

The kit may be provided in a suitable container and/or with suitable packaging.

The kit may include instructions for use, for example, written instructions on how to use the kit in a method for preparing a scaffold for use in 3D tissue culture as set out above.

### Other Preferences

Each and every compatible combination of the embodiments described above is explicitly disclosed herein, as if each and every combination was individually and explicitly recited.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

### Definitions

The term bioink refers to a cell culture composition having appropriate rheological properties to allow it to be printed using 3D printing techniques. Bioinks typically contain a hydrogel component and may optionally contain additional additives, such as growth factors, to influence cell growth and development. Bioinks may contain cells.

The term bioprinting refers to the use of 3D printing techniques to prepare 3D scaffolds for use in tissue culture. Typically, bioprinting techniques deposit a bioink, with or without cells, onto a surface to create a 3D tissue scaffold or a tissue.

The term (meth)acryloyl is used to refer to both an acryloyl group and a methacryloyl group:

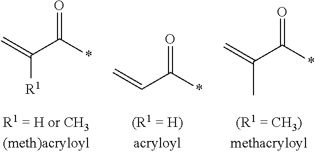

where * represents the attachment point with the remainder of the molecule.

The term (meth)acryloxyl is used to refer to both an acryloxyl group (derived from acrylic acid) and a methacryloxyl group (derived from methacrylic acid). Commonly, the acryloxyl group is also known as an acrylate group and the methacryloxyl group is also known as a methacrylate group. Compounds containing an acryloxyl group may be known as acrylates and compounds containing a methacryloxyl group may be known as methacrylates.

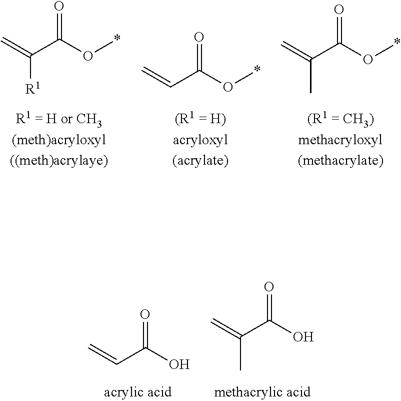

where * represents the attachment point with the remainder of the molecule.

The term (meth)acrylamidyl is used to refer to both an acrylamidyl group (derived from acrylamide) and a methacrylamidyl group (derived from methacrylamide). Commonly, the acrylamidyl group is also known as an acrylamide group and the methacrylamidyl group is also known as a methacrylamide group. Compounds containing an acrylamidyl group may be known as acrylamides and compounds containing a methacrylamidyl group may be known as methacrylamides:

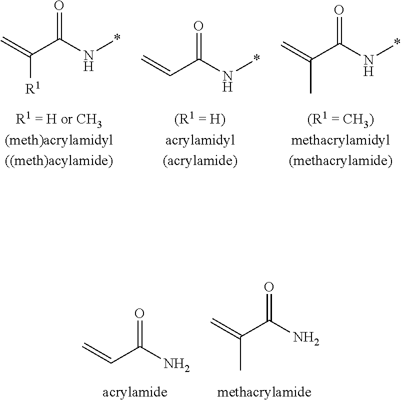

where *represents the attachment point with the remainder of the molecule.

An alkyl group is a monovalent saturated hydrocarbon group. In this context, the prefix (e.g., C₁₋₆) denotes the number of carbon atoms in the hydrocarbon backbone. Alkyl groups may be linear or branched.

An alkylene (alkanediyl) group is a divalent saturated hydrocarbon group in which the two free valencies independently form part of a single bond to separate adjacent atom. In this context, the prefix (e.g. C₁₋₆) denotes the number of atoms in the hydrocarbon backbone. Alkylene groups may be linear or branched.

### EXAMPLES

Certain aspects and embodiments of the invention will now be illustrated by way of example and with reference to the figures described above.

### Experimental Methods

### Example 1: Preparation of Materials

### Example 1-1: PEGylation of Fibronectin with Maleimide-Terminated PEG

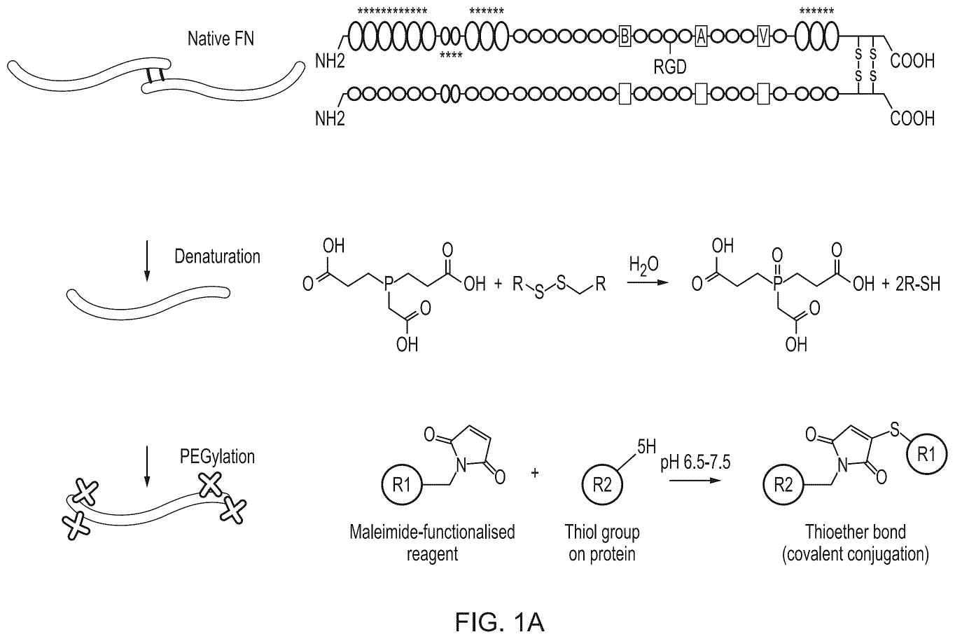

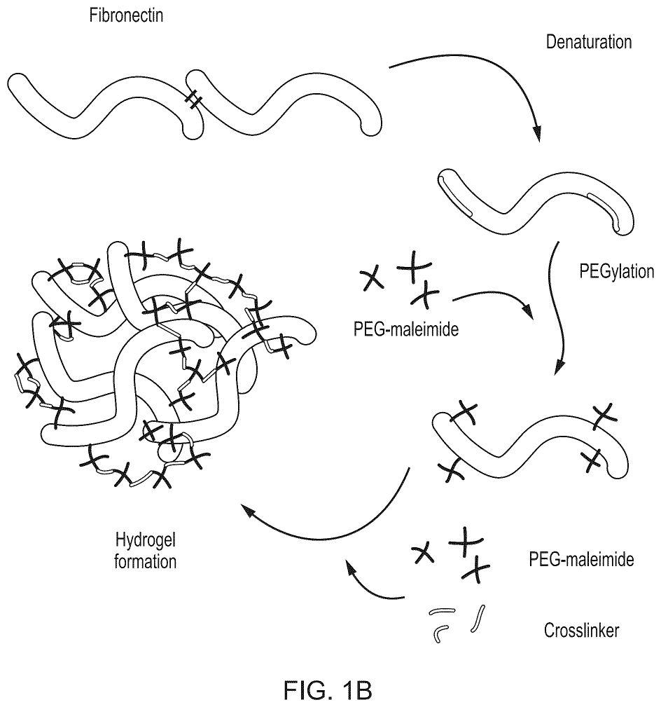

Fibronectin (YOProteins, 3 mg/ml) was PEGylated according to the method of Trujillo et al. Fibronectin was denatured in a denaturing buffer (5 mM Tris [2-carboxyethyl] phosphine hydrochloride [TCEP], pH 7, Sigma) and 8 M urea (Acros Organics, 99.5%) in phosphate buffer saline (PBS, Gibco, pH 7.4) for 15 min at room temperature. Afterwards, 4-arm-PEG-maleimide (PEG-MAL; 20 kDa, LaysanBio) was incubated for 30 min at room temperature at a molar ratio of 1:4 FN to PEGMAL. After obtaining PEGylated protein, non-reacted cysteine residues were blocked by alkylation using 14 mM iodoacetamide (Sigma) in PBS at pH=8. The product of the blocking reaction was dialysed in PBS for 1 h at room temperature. The dialysed protein solution was precipitated by adding 9 volumes of cold absolute ethanol to the protein solution. This was then mixed and incubated overnight at −20° C. The protein solution was centrifuged at 15000 g and 4° C. for 15 min. The supernatant was discarded, and the pellet was washed once more in cold ethanol (90%) and centrifuged at 15000 g and 4° C. for 5 min. Pellets were then dried and dissolved using 8 M urea to provide PEGylated fibronectin (FN PEGylated) with final protein concentration of 2.5 mg/ml. Once the protein was dissolved, the solution was dialysed in PBS one last time and stored at −20° C. for future use. A schematic showing fibronectin denaturation, PEGylation and alkylation is provided in FIG. 1A.

### Example 1-2: PEGylation of Laminins with Maleimide-Terminated PEG

Laminins (Biolamina AB), 100 μg/ml) were PEGylated using MAL-PEG-NHS (5 kDa, Laysan Bio, Inc). Laminin PEGylation was carried out to introduce functional maleimide groups to the protein. LM 411 (579 kDa, Biolamina AB), LM 521 (762 kDa, Biolamina AB), LM 332 (619 kDa, Biolamina AB), and LM 111 (810 kDa, Trevigen Inc.) were PEGylated at a 1:4 molar ratio of LM to PEG-MAL-NHS. A commercial laminin solution in phosphate-buffered saline (PBS) at 100 μg/mL was used. 500 μl of the protein was dialysed to change the buffer to sodium bicarbonate (NaHCO₃; pH 8.5 for PEGylation). To obtain the desired 1:4 molar ratio of AC to LM, calculated volumes of the MAL-PEG-NHS (1 mg/ml in PBS) were added to the respective LM solution and left to mix for 2 h at room temperature. Then, the solution was dialysed to remove all the unreacted maleimides and to exchange the buffer for PBS for 1 h at 4° C. The PEGylated laminins were stored at −20° C.

### Example 1-3: PEGylation of Laminins with Acrylate-Terminated PEG

LMs (Biolamina®), 100 μg/ml) were PEGylated using a bifunctional PEG having terminal acrylate and succinimidyl carboxymethyl ester groups (AC-PEG-SCM; 5 kDa, Laysan Bio, Inc®). LM PEGylation was carried out to introduce functional acrylate groups to the protein. LM 411 (579 kDa, Biolamina®), LM 521 (762 kDa, Biolamina®), LM 332 (619 kDa, Biolamina®) and LM 111 (810 kDa, Trevigen®) were PEGylated according to their respective molecular weights. The PEGylation was performed at a 1:25 molar ratio of LM to PEG-AC-SCM. A commercial solution of the laminins in phosphate-buffered saline (PBS) solution at 100 μg/ml was used. Thus, 500 μl of the proteins were dialysed to change the buffer to sodium bicarbonate (NaHCO₃; pH 8.5 for PEGylation). To obtain the desired 1:25 molar ratio of AC to LM, calculated volumes of the AC-PEG-SCM (1 mg/ml in PBS) were added to the respective LM solution and left to mix for 2 h at room temperature. Then, the solution was dialysed to remove all the unreacted acrylates and change the buffer to PBS for 1 h at 4° C. The LM PEGylated was stored at −20° C.

### Example 1-4: Preparation of GelMA

GelMA was synthesized by conjugating methacrylate functional groups to gelatin according to the method of Loessner et al.

Briefly, gelatin was dissolved in water at 50° C. to a final concentration of 10 wt %. Methacrylic anhydride (MA) was added to the solution dropwise while stirring, at a ratio of 0.6 mL MA per 1 g gelatin. After 3 h of reaction at 50° C., the solution was centrifuged at 3500 g for 3 min and the supernatant, containing the GelMA, was collected and diluted with four volume equivalents of water. The GelMA solution was subsequently dialyzed (12-14 kDa molecular weight cutoff) against water at 40° C. for one week, with the water changed twice a day. This was followed by pH adjustment to 7.4 using 1 m NaHCO₃ and filter sterilization using 0.2 μm vacuum filtration and then freeze drying. All the freeze-dried final products were stored at-20° C.

¹H-NMR spectroscopy was used to confirm the successful conjugation of methacrylate groups, while a fluoraldehyde assay was used to quantify the degree of modification (see Ouyang et al., 2019). Briefly, GelMA samples and gelatin standards were prepared at a concentration range from 0.10-0.75 mg/ml. These solutions were then thoroughly mixed with fluoraldehyde reagent solution (Thermo Fisher) at a volume ratio of 1:2. Triplicate aliquots of each sample and standard were transferred to an opaque 96-well plate (250 μL per well). The fluorescence intensity was determined at 450 nm with an excitation wavelength of 360 nm using a microplate reader (SpectraMax M5, Molecular Devices). The degree of functionalization was approx. 80%.

### Example 2: Preparation of 3D Hydrogels

### Example 2-1: Preparation of 3D GelMA Hydrogels Comprising Encapsulated PEGylated or Native Laminin or Fibronectin

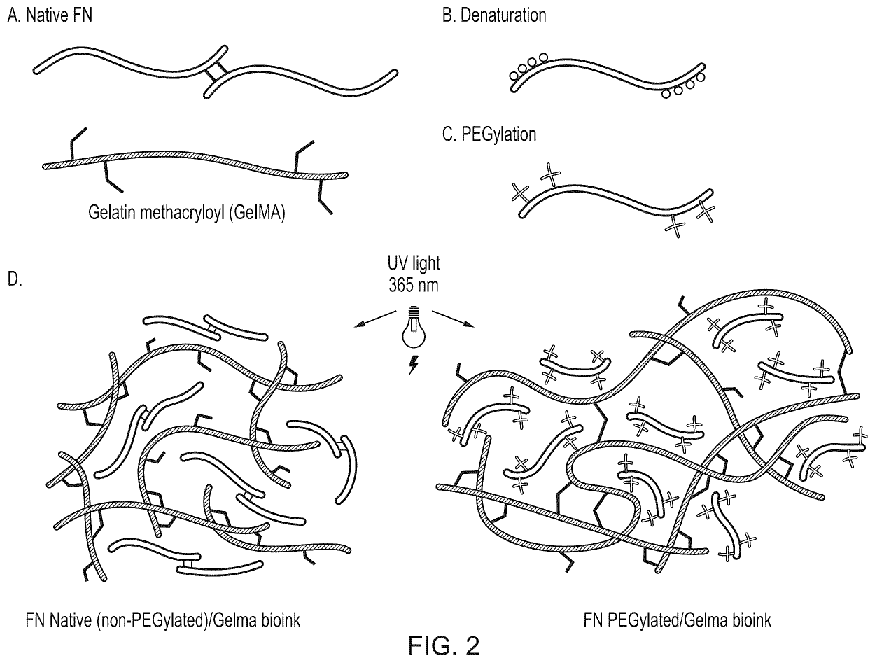

PEGylated or native (non-PEGylated) fibronectin or laminin isoforms (500 μg/ml) was mixed with GelMA and the photoinitiator (Irgacure 2959; 0.05 wt %) to obtain a bioink composition (FIG. 2). The bioink was immediately pipetted into custom sterile polydimethylsiloxane (PDMS) moulds (holes with 6 mm diameter and 3 mm thickness) and polymerised upon exposure to UV light (365 nm, Omnicure S1500, Excelitas Technologies, US) of power ˜5 mW·cm⁻² for 6 minutes to provide GelMA hydrogels comprising encapsulated PEGylated fibronectin (FN PEGylated/GelMA), encapsulated PEGylated laminin (LM PEGylated/GelMA), encapsulated native fibronectin (FN/GelMA), and encapsulated native laminim (LM/GelMA), (e.g., FIG. 2D).

### Comparative Example 2-2: Preparation of 3D PEG-AC Hydrogels Comprising PEGylated or Native Laminin

PEGylated laminin isoforms (Example 1-3) or native laminin (500 μg/ml) were mixed with 4-arm PEG-acrylate (5 wt % PEG-AC, 10 kDa, Laysan Bio, Inc.), protease degradable VMP peptide (GCRDVPMSMRGGDRCG; 1.7 kDa, GenScript) and a photoinitiator (0.1 wt % Irgacure 2959, Sigma-Aldrich) to obtain a bioink for the preparation of degradable hydrogels. To form non-degradable hydrogel, the VPM peptide was substituted with PEG dithiol (SH-PEG-SH, 2 kDa, Creative PEGWorks) at the same volume, as the molecular weights are similar. The thiolated crosslinker was added at a molar ratios of 2:1, 1:1 and 1:2 (acrylate: thiol) to obtain hydrogel mixtures with various degradability. The bioink was immediately pipetted into custom sterile polydimethylsiloxane (PDMS) moulds (holes with 6 mm diameter and 3 mm thickness) and polymerised upon exposure to UV light (365 nm, Omnicure S1500, Excelitas Technologies, US) of power ˜5 mW·cm⁻² for 6 minutes to provide PEG-AC hydrogels comprising PEGylated laminin (LM PEGylated/PEG-AC) or native laminin (LM/PEG-AC). Synthesised hydrogels were then transferred to sterile non-tissue culture treated plate wells.

### Comparative Example 2-3: Preparation of 3D PEG-MAL Hydrogels Comprising Cross-Linked PEGylated Fibronectin or Native Fibronectin

PEG hydrogels were formed using Michael-type addition reaction under physiological pH and temperature according to the method of Phelps et al. (see FIG. 1B). PEGylated or native fibronectin was mixed with PEG maleimide (PEG-MAL) and a thiolated crosslinker, either PEG dithiol (PEG-SH, 2 kDa, Creative PEGWorks) or a mixture of PEG-SH and VMP peptide (purity 96.9%, Mw 1696.96 Da, GenScript), was added at a molar ratio 1:1 maleimide: thiol. After addition of the crosslinker, the samples were incubated for 30 min at 37° C. to allow gelation and provide PEG-MAL hydrogels comprising PEGylated fibronectin (FN PEGylated/PEG-MAL) or native fibronectin (FN/PEG-AC).

Where used, cells and/or soluble molecules such as growth factors were mixed into the FN-PEG and PEG-MAL compositions before addition of the crosslinker.

Where appropriate, PEGylated fibronectin was exchanged

### Comparative Example 2-4: Preparation of 3D PEG-MAL Hydrogels without Protein

PEG-only hydrogels were produced in the same manner as Comparative Example 2-3, without the addition of the PEGylated FN.

The nomenclature used in this document is: x % (FN)-PEG yVPM, x being the percentage of PEGMAL used and y the fraction of degradable crosslinker added. Where not indicated, hydrogels were 5% FN-PEG 0.5VPM.

### Example 3: Release of ECM Proteins from Hydrogels

To investigate the release characteristics of PEGylated and non-PEGylated protein, hydrogels were prepared according to the methods of Examples 2-1 to 2-4. The composition of the hydrogels is set out in Table 1.

TABLE 1

| | Entry | | | | |
|---|---|---|---|---|---|
| Component | 3-1 | 3-2 | 3-3 | 3-4 | 3-5 |
| protein | FN PEGylated | FN PEGylated | FN PEGylated | FN PEGylated | FN PEGylated |
| quantity | 0.5 mg/ml | 0.5 mg/ml | 0.5 mg/ml | 0.5 mg/ml | 0.5 mg/ml |
| polymer | GelMA | GelMA | PEGAC | PEGMAL | PEGMAL |
| quantity | 5 wt % | 5 wt % | 5 wt % | 5 wt % | 5 wt % |
| cross-linker | — | — | PEG-Dithiol | PEG-Dithiol | — |
| quantity | — | — | 1.4 wt % | 0.82 wt % | — |
| photoinitiator | Irgacure 2959 | Irgacure 2959 | Irgacure 2959 | Irgacure 2959 | Irgacure 2959 |
| quantity | 0.05 wt % | 0.05 wt % | 0.05 wt % | 0.05 wt % | 0.05 wt % |

To determine the mass percentage release of fibronectin or laminin from native fibronectin or laminin PEGylated/(GelMA or PEGMAL) or LM PEGylated/PEGAC hydrogels, BMP-2 was conjugated to with NHS-AlexaFluor-488 dye (10 μg mL-1, Gibco-Life Technologies) to allow its detection using a fluorescence plate reader. Synthesised hydrogels were transferred to individual centrifuge tube filters (Eppendorfs with filter units, Corning Costar spin-X) loaded with PBS as a releasing buffer. Over 5 days, the tubes containing the hydrogels mixed with fibronectin or laminin were centrifuged at a rate of 8000 rpm for 10 min once per day. The supernatant was collected and stored at −20° C. and the filter units were reloaded with 400 μL of PBS. Ultimately the supernatant aliquots were pipetted into a black non-tissue culture treated plate and the fluorescence intensity of the GF released from the hydrogels was measured at 490 nm excitation and 510-570 nm emission wavelength using a microplate reader (Modulus II Microplate Multimode Reader, Turner BioSystems). The concentration of fibronectin or laminin in the samples was quantified using a standard curve, which relates fluorescence intensity to fibronectin or laminin concentration, obtained from serial dilution of fibronectin or laminin solutions.

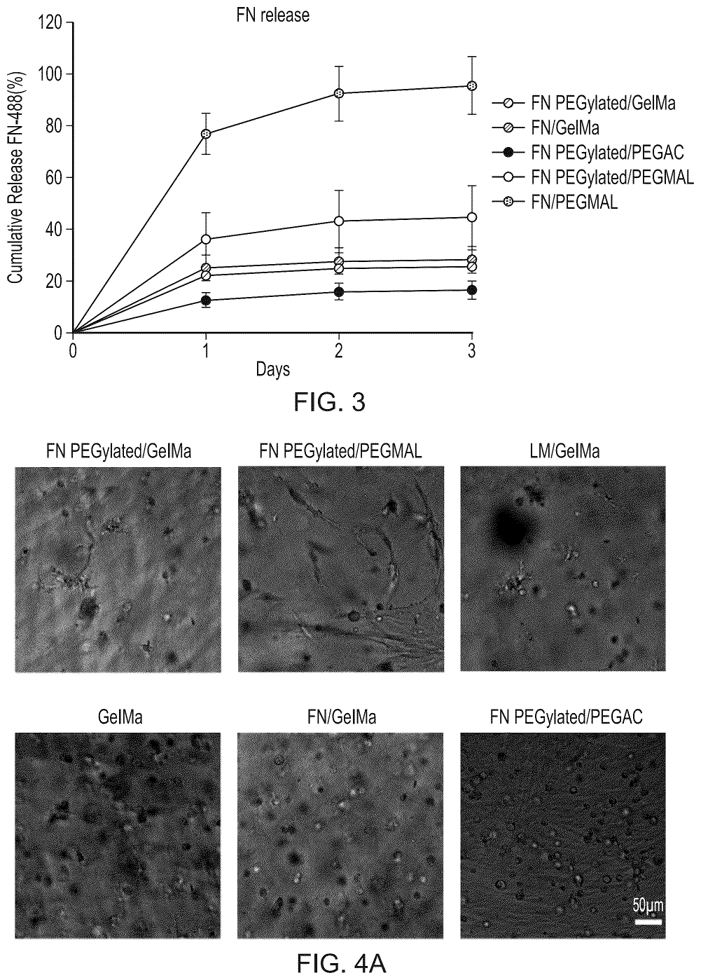

As shown in FIG. 3, the release of PEGylated fibronectin from the GelMA hydrogel was slower than the release of PEGylated fibronectin from PEGMAL hydrogel. This is surprising, as the PEG maleimide derivatised fibronectin is merely physically entrapped within, and not chemically cross-linked to, the GelMA hydrogel. In contrast, the PEGylated fibronectin is chemically cross-linked into the PEGMAL hydrogel.

If fibronectin is covalently crosslinked into the PEG or GelMa, the fibronectin should be retained in the hydrogel and release of fibronectin should be minimal. On the other hand, if native fibronectin is added to the hydrogel or bioink system, as the is no chemical crosslink with the hydrogel, it should be release by diffusion.

Also, PEGylated fibronectin embedded in PEGAC showed the same retention as fibronectin in the GelMA gels due to the smaller mesh size of the hydrogel. As expected, non-crosslinked fibronectin in PEGMAL gels shows a very fast release kinetics.

### Example 4: Vascularisation of HUVECs and HDFs in Hydrogels?

To investigate the potential of hydrogels comprising PEGylated and non-PEGylated protein to promote vascularisation, hydrogels containing encapsulated HUVECs and HDFs were prepared according to the methods of Examples 2-1 to 2-4. The composition of the hydrogels is set out in Table 2.

TABLE 2

| | Entry | | | | | | | | | | | |
|---|---|---|---|---|---|---|---|---|---|---|---|---|
| Component | 4-1 | | 4-2 | | 4-3 | | 4-4 | | 4-5 | | 4-6 | |
| protein | FN | | FN | | LM | | — | | FN native | | FN | |
| | PEGylated | | PEGylated | | | | | | | | PEGylated | |
| quantity | 500 | μg/ml | 500 | μg/ml | 500 | μg/ml | — | | 500 | μg/ml | 500 | μg/ml |
| polymer | GelMA | | PEGMAL | | GelMA | | GelMA | | GelMA | | PEGAC | |
| quantity | 5 | wt % | 5 | wt % | 5 | wt % | 5 | wt % | 5 | wt % | 5 | wt % |
| cross-linker | — | | VPM | | — | | — | | — | | VPM | |
| quantity | | | 0.82 | wt % | | | | | | | 1.4 | wt % |
| photoinitiator | Irgacure | | Irgacure | | Irgacure | | Irgacure | | Irgacure | | Irgacure | |
| | 2959 | | 2959 | | 2959 | | 2959 | | 2959 | | 2959 | |
| quantity | 0.05 | wt % | 0.05 | wt % | 0.05 | wt % | 0.05 | wt % | 0.05 | wt % | 0.05 | wt % |
| cells | HDF and | | HDF and | | HDF and | | HDF and | | HDF and | | HDF and | |
| | HUVECs | | HUVECs | | HUVECs | | HUVECs | | HUVECs | | HUVECs | |
| quantity | 1 × 10⁶ | per ml | 1 × 10⁶ | per ml | 1 × 10⁶ | per ml | 1 × 10⁶ | per ml | 1 × 10⁶ | per ml | 1 × 10⁶ | per ml |

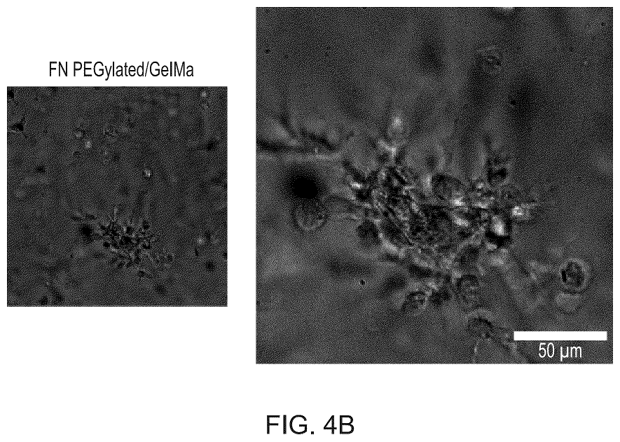

Brightfield images were taken at day 9 using a Zeiss AxioObserver inverted epifluorescence microscope with an Andor cooled CCD camera, 1 MP, monochromatic (FIG. 4A). FN PEGylated/GelMA bioink showed high levels of sprouting after 7 days as shown in FIG. 4B.

The inventors believe that hydrogels comprising PEGylated fibronectin encapsulated within GelMa encourage vascular tubular formation due to the exposure of the FN domains (e.g. cell and growth factors binding) after PEGylation and the higher retention of PEGylated FN in the GelMa from the release experiment.

Other extracellular matrix proteins such as fibronectin or collagen can be pegylated and mixed with GelMa or hyaluronic acid methacrylate, alginate combined with collagen or gelatin to regenerate different tissue such as bone, nerve or skin using the scaffolds of the invention.

### Example 5: 3D Bioprinting of Hydrogels

The bioink mixture can be printed using a multi-nozzle 3D Discovery bioprinter (RegenHu, Switzerland)-Glasgow where the temperature of both the extrusion nozzle and print bed can be controlled. The print bed was set to around 17° C., which triggers the thermal gelation of gelatin-based hydrogels after printing. The nozzle temperature was set at values that would result in smooth gel filament extrusion. The printing of templating and matrix bioinks can be done side by side in a layer-by-layer fashion. HUVECs and HDFs can be printed in parallel layer using two printing heads. The PEGMAL, PEGAC and GelMA combine with FN PEGylated or non-PEGylated were mixed with 1×10⁶ HUVECs per ml and 1×10⁶ HDF per ml to assess the in-situ angiogenesis. The matrix bioink comprised different photo-crosslinkable formulations, including: 5 wt % GelMA and 500 μg/ml FN PEGylated, 5 wt % GelMA and 500 μg/ml FN non-PEGylated, 5 wt % GelMA, 5 wt % GelMA with 4-arm PEG-acrylate (PEGAC, 10 kDa, Laysan Bio, Inc.) and 500 μg/ml FN PEGylated, 5 wt % PEGMAL and 500 μg/ml FN PEGylated and 5 wt % PEGMAL and 500 μg/ml FN non-PEGylated.

### REFERENCES

A number of publications are cited above in order to more fully describe and disclose the invention and the state of the art to which the invention pertains. Full citations for these references are provided below. The entirety of each of these references is incorporated herein.
Dobre et al, “A Hydrogel Platform that Incorporates Laminin Isoforms for Efficient Presentation of Growth Factors—Neural Growth and Osteogenesis”, Adv. Funct. Mater., 2021, Vol. 31, pp. 0210225.
Doi et al., “Recombinant Human Laminin-10 (α5β1γ1)”, Journal of Biological Chemistry, 2002, Vol. 277, pp. 12741-12748.
Francisco et al., “Photocrosslinkable laminin-functionalized polyethylene glycol hydrogel for intervertebral disc regeneration”, Acta Biomater., 2014, Vol. 10, pp. 1102-1111.
Loessner et al., “Functionalization, preparation and use of cell-laden gelatin methacryloyl-based hydrogels as modular tissue culture platforms”, Nat. Protoc. 2016, Vol. 11, pp. 727-746.
Ouyang et al., “Void-Free 3D Bioprinting for In Situ Endothelialization and Microfluidic Perfusion”, Adv. Funct. Mater., 2019, Vol. 30, pp. 1908349.
Phelps et al., “ ” Bioartificial matrices for therapeutic vascularization, Proc. Natl. Acad. Sci. U.S.A., 2010, Vol. 107, pp. 3323-3328.
US 2006/0233855 A1
Seidlits et al., “Fibronectin-hyaluronic acid composite hydrogels for three-dimensional endothelial cell culture”, Acta Biomater., 2011, Vol. 7, pp. 2401-2409.
Trujillo et al., “Engineered Full-Length Fibronectin—Hyaluronic Acid Hydrogels for Stem Cell Engineering”, Adv. Healthcare Mater., 2020, Vol. 9, 2000989.
Trujillo et al., “Engineered full-length Fibronectin-based hydrogels sequester and present growth factors to promote regenerative responses in vitro and in vivo”, bioRxiv, 2019, 687244.

1. A composition for use in 3D tissue culture comprising:
(a) a biocompatible polymer suitable for the preparation of a hydrogel selected from polypeptides, polysaccharides and synthetic polymers; and
(b) a modified extracellular matrix (ECM) protein, or a fragment thereof, comprising a group:
-L-B
wherein -L- is a synthetic linker and —B is a blocking group that is unreactive towards the biocompatible polymer such that, after preparation of a hydrogel, the modified ECM protein is not covalently bound to the hydrogel.

2. The composition of claim 1, wherein the biocompatible polymer is gelatin methacryloyl (GelMA).

3. The composition of 1 or 2, wherein the ECM protein is selected from laminin, fibronectin, and fragments of these.

4. The composition of claim 3, wherein the ECM protein is:
i) a laminin isoform selected from LM521, LM332; or
ii) a laminin-derived peptide selected from IKVAV and YIGSR; or
iii) a fragment of fibronectin in comprising any one of FNI₁₋₅, FNIII₁₋₂, FNIII₇, FNIII₉₋₁₀, FNIII₁₀, FNIII₁₂₋₁₄, and FNIII₁₂₋₁₅; or
iv) the fibronectin derived peptide GRGDSPC; or
v) full-length fibronectin.

5. The composition of any preceding claim, wherein the synthetic linker-L-comprises polyethylene glycol (PEG).

6. The composition of claim 5, wherein the PEG comprises a 4-arm PEG.

7. The composition of any preceding claim, wherein the blocking group-B is selected from hydrogen, C₁₋₆ alkyl, carboxylic acid, amine and maleimide.

8. The composition of claim 7, wherein the blocking group-B is maleimide: 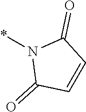 [SMILES: *N1C(=O)C=CC1=O]
where * represents the attachment point with the synthetic linker -L-.

9. The composition of any preceding claim comprising mammalian cells.

10. The composition of any preceding claim comprising a growth factor and/or cytokine.

11. The composition of any preceding claim comprising a photoinitiator, such as a photoinitiator selected from Irgacure 2959, LAP, VA-086 and eosine Y.

12. The composition of any preceding claim comprising phosphate-buffered saline.

13. A 3D scaffold obtained or obtainable by polymerisation of the composition of any preceding claim.

14. A scaffold for use in 3D tissue culture comprising:
(a) a polypeptide, polysaccharide or synthetic hydrogel obtained or obtainable by polymerisation of a biocompatible polymer; and
(b) a modified extracellular matrix (ECM) protein, or a fragment thereof, comprising a group:
-L-B
wherein -L- is a linker and —B is a blocking group that is unreactive towards the biocompatible polymer, and wherein the modified ECM protein is not covalently bound to the hydrogel.

15. The scaffold of claim 14, wherein the hydrogel is a GelMA-based hydrogel.

16. The scaffold of claim 14 or 15, wherein the ECM protein is selected from laminin, fibronectin, and fragments of these.

17. The scaffold of any of claims 14 to 16, wherein the synthetic linker -L- comprises PEG.

18. The scaffold of any of claims 14 to 17, wherein the blocking group —B is selected from hydrogen, C₁₋₆ alkyl, carboxylic acid, amine and maleimide.

19. The scaffold of any of claims 14 to 18, comprising mammalian cells.

20. A method for preparing a scaffold for use in 3D tissue culture, the method comprising:
(a) providing a composition of any one of claims 1 to 12;
(b) extruding the composition through a print nozzle onto a print bed to provide a 3D structure; and
(c) curing the 3D structure.

21. The method of claim 20, wherein:
(b) further comprises extruding a templating composition through a print nozzle onto the print bed to provide a 3D structure comprising the composition of any one of claims 1 to 12 and the templating composition;
and optionally the method comprises:
(d) liquifying and removing the cured templating composition.

22. The method of claim 20 or 21, wherein (c) comprises irradiating the sample with light.

23. A 3D scaffold obtained or obtainable by the method of any one of claims 20 to 22.

24. A method for preparing a tissue, the method comprising:
(a) providing a scaffold of any one of claims 13 to 19 and 23, wherein the scaffold comprises mammalian cells;
(b) culturing the scaffold under physiological conditions.

25. A tissue obtained or obtainable by the method of claim 24.
